(19) United States
(12) Patent Application Publication
(10) Pub. No.: US 2020/0166506 A1
(43) Pub. Date: May 28, 2020

(54) MODIFIED NANOPARTICLE, DISPERSION CONTAINING MODIFIED NANOPARTICLE, SET FOR RESISTIVE PULSE SENSING, SET AND REAGENT FOR DETECTING VIRUS OR BACTERIUM, AND METHOD FOR DETECTING VIRUS OR BACTERIUM

(71) Applicant: National University Corporation Tokyo Medical and Dental University, Bunkyo-ku, Tokyo (JP)

(72) Inventors: Yuji Miyahara, Tokyo (JP); Akira Matsumoto, Tokyo (JP); Tatsuro Goda, Tokyo (JP); Yukichi Horiguchi, Tokyo (JP)

(73) Assignee: National University Corporation Tokyo Medical and Dental University, Bunkyo-ku, Tokyo (JP)

(21) Appl. No.: 16/608,609

(22) PCT Filed: Apr. 25, 2018
(86) PCT No.: PCT/JP2018/016851

§ 371 Date: Feb. 11, 2020

(30) Foreign Application Priority Data
Apr. 28, 2017 (JP) 2017-090567

(51) Int. Cl. G01N 33/543; G01N 27/12; G01N 1/38; G01N 33/569; G01N 33/553

(52) U.S. Cl. CPC G01N 33/54346; G01N 27/127; G01N 33/553; G01N 33/56911; G01N 33/56983; G01N 1/38

(57) ABSTRACT

A modified nanoparticle includes a nanoparticle, a dispersibility improving group bound to a surface of the nanoparticle, and an oligosaccharide that is bound to the surface of the nanoparticle, and that selectively captures a specific virus or bacterium. A reagent for detection of a specific virus or bacterium by resistive pulse sensing is also provided, the reagent including the modified nanoparticle.

### TECHNICAL FIELD

The present disclosure relates to a modified nanoparticle, a dispersion liquid containing the modified nanoparticle, a set for resistive pulse sensing, a set and a reagent for detecting a virus or bacterium, and a method of detecting a virus or a bacterium.

### BACKGROUND ART

Infectious diseases are social problems, and in order to overcome the problems, it is necessary to quickly determine whether or not infection has occurred.

There is a method using a biosensor as a method for quickly monitoring whether or not virus or a bacterium infection has occurred. The biosensor is desired to have capacities to monitor many kinds of viruses or bacteria easily and with higher sensitivity.

As a method of detecting an influenza virus, Japanese Patent Application Laid-Open (JP-A) No. 2014-095720 describes a method of detecting an influenza virus subtype H5 based on an immunoassay using an antibody against a hemagglutinin protein of an influenza virus subtype H5.

Meanwhile, as means for detecting hemagglutinin of an influenza virus, a detection method using a device in which an oligosaccharide is immobilized on a gate insulator film of a field effect transistor (Anal. Chem. 2013, 85, 5641-5644) has also been reported.

Japanese Patent Application Laid-Open (JP-A) No. 2014-169964 discloses a method of producing a sensor chip in which a sugar chain-containing compound, having a sugar chain that specifically binds to a protein or biotoxin derived from a bacterium or virus, is immobilized on a substrate surface on which gold nanoparticles are immobilized. Japanese Patent Application Laid-Open (JP-A) No. 2011-209282 discloses a method of producing a sugar chain-immobilized fluorescent nanoparticle, the method including binding a ligand complex, constituted with a sugar chain and a linker compound bonded together, to a heat-treated fluorescent nanoparticle to obtain a fluorescent nanoparticle on which the sugar chain is immobilized.

Study to detect a substance using particles is also underway. Japanese Patent Application Laid-Open (JP-A) No. 2016-126003, Applied Physics Letters, vol. 108 (2016), p. 123701: 1-5, and Small, vol. 2 (Wiley-VCH Verlag GmbH & Co, 2006), No. 8-9, p. 967-972 describe detection of existence of a substance of interest in a specimen by observation of changes in electric current when a particle modified with an antibody passes a through hole.

### SUMMARY OF INVENTION

### Technical Problem

It is known that some viruses and bacteria specifically recognize an oligosaccharide present on the cell surface of a host to develop infection. The inventors of the present invention have found that, when modified nanoparticles having a surface to which the oligosaccharide and a dispersibility improving group are bound are used, the nanoparticles can be made to specifically attach to a desired virus or bacterium through the oligosaccharide. The inventors have also found that the target virus or bacterium can be selectively detected by detecting a dimensional change of the virus or bacterium caused by attachment of the modified nanoparticles using resistive pulse sensing.

The present disclosure is based on the above findings, and provides a modified nanoparticle capable of selectively attaching to a virus or bacterium, a dispersion liquid containing the modified nanoparticle, a set for resistive pulse sensing that includes the modified nanoparticle or the dispersion liquid, a set for detection of a specific virus or bacterium that includes the modified nanoparticle or the dispersion liquid, a reagent capable of selectively and highly sensitively detecting a specific virus or bacterium by resistive pulse sensing, and a method of detecting a virus or bacterium using the reagent.

### Solution to Problem

Aspects of the present disclosure include the following <1> to <14>.
<1> A modified nanoparticle, including:

a nanoparticle;

a dispersibility improving group bound to a surface of the nanoparticle; and

an oligosaccharide that is bound to the surface of the nanoparticle, and that selectively captures a specific virus or bacterium.
<2> The modified nanoparticle according to <1>, wherein the nanoparticle is a metal nanoparticle or a polymer nanoparticle.
<3> The modified nanoparticle according to <1> or <2>, wherein a number average particle size of the nanoparticle is from 5 nm to 100 nm.
<4> The modified nanoparticle according to any one of <1> to <3>, wherein the oligosaccharide selectively captures an influenza virus.
<5> The modified nanoparticle according to <4>, wherein the oligosaccharide selectively captures a specific type of influenza virus.
<6> The modified nanoparticle according to any one of <1> to <5>, wherein the dispersibility improving group has, at a terminal thereof, a sulfobetaine group, a carboxybetaine group, or a phosphobetaine group.
<7> A dispersion liquid, including the modified nanoparticle according to any one of <1> to <6> and an aqueous medium.
<8> A set for resistive pulse sensing, the set including the modified nanoparticle according to any one of <1> to <6>, or the dispersion liquid according to <7>, and a pore-containing membrane for resistive pulse sensing.
<9> A set for detection of a specific virus or bacterium, the set including the modified nanoparticle according to any one of <1> to <6> or the dispersion liquid according to <7>, and a resistive pulse sensing device.
<10> A reagent for detection of a specific virus or bacterium by resistive pulse sensing, the reagent including the modified nanoparticle according to any one of <1> to <6>.
<11> The reagent according to <10>, for detecting the specific virus or bacterium based on presence or absence of a shift of a particle size peak in a particle size distribution.
<12> A method of detecting a virus or bacterium, the method including:

(a) a step of measuring a particle size distribution of particles included in a biological liquid sample by resistive pulse sensing;

(b) a step of preparing a mixed liquid by mixing the biological liquid sample with the reagent according to <10> or <11>; and

(c) a step of measuring a particle size distribution of particles included in the mixed liquid by resistive pulse sensing,

wherein the biological liquid sample is judged to include the virus or bacterium when there is a peak in a particle size range corresponding to the virus or bacterium, of which a peak position in the particle size distribution obtained in the step (c) exhibits a shift toward a larger particle size side, compared to a peak position thereof in the particle size distribution measured in the step (a).

<13> The detection method according to <12>, wherein the biological liquid sample is mixed with a nanoparticle not having the oligosaccharide on a surface thereof, before the measurement by resistive pulse sensing in the step (a).

<14> The detection method according to <12> or <13>, wherein the biological liquid sample is mixed with an aqueous medium in at least one of the step (a) or the step (b).

### Advantageous Effects of Invention

According to aspects of the present disclosure, a modified nanoparticle capable of selectively attaching to a virus or bacterium, a dispersion liquid containing the modified nanoparticle, a set for resistive pulse sensing that includes the modified nanoparticle or the dispersion liquid, a set for detection of a specific virus or bacterium that includes the modified nanoparticle or the dispersion liquid, a reagent capable of selectively and highly sensitively detecting a specific virus or bacterium by resistive pulse sensing, and a method of detecting a virus or bacterium using the reagent can be provided.

### BRIEF DESCRIPTION OF DRAWINGS

FIG 1A is a schematic diagram illustrating a state in which a sample including virus particles is measured by resistive pulse sensing.

FIG. 1B is a schematic diagram illustrating a state in which virus particles having nanoparticles attached thereto are measured by resistive pulse sensing, the nanoparticles (molecular recognition nanoparticles) having, on a surface thereof, an oligosaccharide that selectively captures a specific virus or bacterium.

FIG. 1C is a diagram illustrating a movement (shift) of the particle size peak occurring between before and after molecular recognition.

FIG. 2 is a process diagram illustrating preparation of 6′SLN-GNP from tetrachloroauric (III) acid.

FIG. 3A is a scatter plot of duration vs. particle size obtained from the results of resistive pulse sensing measurement of a virus solution.

FIG. 3B is a scatter plot of duration vs. particle size obtained from the results of resistive pulse sensing measurement of a virus solution with which 6′SLN-GNP has been mixed.

FIG. 3C is a scatter plot of duration vs. particle size obtained from the results of resistive pulse sensing measurement of a virus solution with which 3′SLN-GNP has been mixed.

FIG. 4 is histograms indicating the number of particles in each particle size bin obtained by converting the scatter plots of FIGS. 3A to 3C. The ordinate represents the relative value of the number of particles (normalized based on the maximum value).

FIG. 5 is histograms indicating the number of particles in each duration (duration of an electrical resistance increase peak) bin obtained by converting the scatter plots of FIGS. 3A to 3C. The ordinate represents the relative value of the number of particles (normalized based on the maximum value).

FIG. 6 is an experimental result showing the presence or absence of aggregation when a nanoparticle solution is concentrated using a rotary evaporator. The % in the figure represents the molar proportions of MUA and SB-SH used.

FIG. 7 is histograms indicating the relative value of the number of particles (ordinate: normalized based on the maximum value) vs. particle size (abscissa) showing the results of molecular recognition experiments with respect to influenza virus type A subtype H1N1.

FIG. 8 is a diagram showing the waveform separation of a histogram indicating the relative value of the number of particles (ordinate: normalized based on the maximum value) vs. particle size (abscissa) obtained at the time of measurement of an influenza virus by resistive pulse sensing.

### DESCRIPTION OF EMBODIMENTS

Various embodiments according to the present disclosure will be specifically described below while explaining components and process steps used in the present disclosure.

The scope of the term “step” as used herein includes not only an independent step, but also a step that is not clearly separated from another step, insofar as an intended function of the step can be attained.

In the present disclosure, each numerical range expressed by “from x to y” indicates a range that includes x and y as the minimum and maximum values, respectively.

In the present disclosure, when the content of a component in a composition is indicated, and plural substances that each read on the component are present in the composition, the content refers to the total amount of the plural substances present in the composition, unless otherwise specified.

According to the present disclosure, a modified nanoparticle capable of selectively attaching to a virus or bacterium, a dispersion liquid containing the modified nanoparticle, a set for resistive pulse sensing that includes the modified nanoparticle or the dispersion liquid, a set for detection of a specific virus or bacterium that includes the modified nanoparticle or the dispersion liquid, a reagent capable of selectively and highly sensitively detecting a specific virus or bacterium by resistive pulse sensing, and a method of detecting a virus or bacterium using the reagent are provided. Embodiments according to the present disclosure will be specifically described below.

### Modified Nanoparticle

A modified nanoparticle according to the present disclosure includes a nanoparticle, a dispersion liquid improving group bound to a surface of the nanoparticle, and an oligosaccharide that is bound to the surface of the nanoparticle, and that selectively captures a specific virus or bacterium.

A modified nanoparticle according to the present disclosure can highly selectively attach to a specific virus or bacterium. This is presumably for the following reasons.

In a modified nanoparticle according to the present disclosure, an oligosaccharide that selectively captures a specific virus or bacterium has been bonded to the surface of the nanoparticle. Nanoparticles having a surface having the oligosaccharide, which selectively captures a specific virus or bacterium, bonded thereto attach selectively to a specific virus or bacterium (hereinafter also referred to as detection target). However, the inventors have found that, in practice, when nanoparticles, of which the surface has only the oligosaccharide that selectively captures a detection target, are used, the nanoparticles also attach to coexisting substances other than the detection target, especially to a structure having a similar structure to the detection target, and therefore the selectivity of attachment decreases. This is conceivably because the dispersion stability of the nanoparticles is not sufficient. The inventors have found that, in the modified nanoparticle according to the present disclosure, since a dispersibility improving group as well as the oligosaccharide that selectively captures a detection target are bonded to the surface of the nanoparticle, attachment of the resultant nanoparticle (modified nanoparticle) to coexisting substances is reduced, and a higher selectivity in attachment of the modified nanoparticle to a detection target can be realized. This is conceivably due to improved dispersion stability of the modified nanoparticle.

As discussed above, the modified nanoparticle according to the present disclosure can selectively attach to a specific virus or bacterium that is a detection target. Therefore it is possible to detect the presence of the detection target and/or to measure the quantity of the detection target by detecting the attachment of the modified nanoparticle or the quantity thereof.

### Nanoparticle

Nanoparticles used in the modified nanoparticles according to the present disclosure may be any particles having an average particle size of less than 1 μm, and are preferably particles having an average particle size of 500 nm or less. In this regard, the average particle size of nanoparticles means the number average value (number average particle size) of the maximum diameter of each of 100 particles observed under a transmission electron microscope. The transmission electron microscope for measurement is, for example, JEM-2100P manufactured by JEOL Ltd.

When the average particle size of the particles is 1 μm or more (microparticles), the ratio of the area of contact with the detection target is small relative to the particle size, and attachment to the detection target is apt to be unstable. The lower limit of the average particle size of the nanoparticles may be, for example, 5 nm. When the particle size of the nanoparticles is too small, attachment of the modified nanoparticles, which are produced from the nanoparticles, to the detection target does not make the size of a complex of the detection target and the modified nanoparticles attached to the surface of the detection target (hereinafter sometimes referred to as a “detection target-modified nanoparticles complex”) significantly larger than the size of the detection target itself, and, therefore, detection tends to be difficult in the case of using a technique whereby attachment is detected based on the size change.

The average particle size of the nanoparticles may be, for example, in a range of from 5 nm to 200 nm, in a range of from 5 nm to 100 nm, in a range of from 10 nm to 100 nm, or in a range of from 15 nm to 50 nm. An appropriate size of the nanoparticles may be set in consideration of the size and/or shape of the detection target. The average particle size of the nanoparticles may be, for example, from 1% to 10%, from 5% to 50%, or from 10% to 30% of the maximum length of the detection target. When the average particle size of the nanoparticles is within the foregoing ranges, attachment of the nanoparticles to the detection target can be clearly detected based on a change in the size or the like while the nanoparticles can be stably attached to the detection target. When the average particle size of the nanoparticles is too small, detection of attachment of the nanoparticles tends to be more difficult.

The particle sizes of the nanoparticles are preferably monodisperse from the viewpoint of detecting the attachment of the modified nanoparticles to the detection target with high reliability. Further, the full width at half maximum of the peak of the particle size distribution of the nanoparticles is preferably 50% or less of the average particle size of the nanoparticles, more preferably 30% or less of the average particle size of the nanoparticles, and further preferably 10% or less of the average particle size of the nanoparticles, from the viewpoint of detecting the attachment of the modified nanoparticles to the detection target with high reliability.

There is no particular restriction on the shapes of the nanoparticles, and examples thereof include a spherical shape, a columnar shape, and a spheroid shape. From the viewpoint of reducing unevenness of the properties at different particle orientations, a spherical shape or a nearly spherical shape is preferable. For example, the value of the Wadell's practical sphericity Ψw obtained from the following Formula (the average value of the Ψw values of the particles) is preferably 0.9 or more, more preferably 0.95 or more, and further preferably 0.98 or more. In the case of a perfect sphere, Ψw is 1. Therefore, the maximum value of Ψw is theoretically 1.

Sphericity=(circumference of circle having the same projected area)/(circumference of particle)

There is no particular restriction on the material configuring the nanoparticles, and the nanoparticles may be metal nanoparticles, polymer nanoparticles, or nanoparticles made of another material. Examples of the metal nanoparticles include Au nanoparticles, Ag nanoparticles, Zn nanoparticles, Al nanoparticles, Co nanoparticles, Cu nanoparticles, Sn nanoparticles, Ta nanoparticles, Ti nanoparticles, Fe nanoparticles, Ni nanoparticles, Pd nanoparticles, and Mo nanoparticles. The metal nanoparticles may alternatively be alloy nanoparticles, for example, Ag—Cu nanoparticles, As—Sn nanoparticles, Cu—Zn nanoparticles, Fe—Ni nanoparticles, or the like. Examples of the polymer particles include polystyrene nanoparticles, poly(methyl acrylate) nanoparticles, poly(methyl methacrylate) nanoparticles, and fluorocarbon polymer nanoparticles. Examples of the nanoparticles made of another material include nanoparticles of a metal oxide, carbon nanoparticles, and diamond nanoparticles. Examples of the metal oxide nanoparticles include calcium oxide nanoparticles, calcium phosphate nanoparticles, hydroxyapatite nanoparticles, cerium(IV) oxide nanoparticles, cobalt(II or III) oxide nanoparticles, chromium(III) oxide nanoparticles, copper(I or II) oxide nanoparticles, iron(II or III) oxide nanoparticles, indium(III) oxide nanoparticles, magnesium oxide nanoparticles, molybdenum(IV) oxide nanoparticles, silica nanoparticles, tin(IV) oxide nanoparticles, Ti(IV) oxide nanoparticles, zinc oxide nanoparticles, and zirconium(IV) oxide nanoparticles. These materials are available from Sigma-Aldrich, Inc.

Among these, Au nanoparticles and polystyrene nanoparticles are preferable, and Au nanoparticles are more preferable, from the viewpoints of the stability and the suitability for surface modification.

The nanoparticles may be obtained as a commercial product having a uniform particle size as exemplified above, or alternatively obtained by performing a nanoparticle generation reaction. For example, in the case of gold nanoparticles, the gold nanoparticles can be prepared by reducing tetrachloroauric(III) acid, and, for example, NaBH₄ may be used as a reducing agent.

### Oligosaccharide That Selectively Captures Specific Virus or Bacterium

In the modified nanoparticle according to the present disclosure, an oligosaccharide that selectively captures a specific virus or bacterium is bound to the surface of the nanoparticle. In the present disclosure, the bond between the surface of the nanoparticle and the oligosaccharide that selectively captures a specific virus or bacterium is not limited to a mode in which the surface and the oligosaccharide are directly bound, but also includes a mode in which the surface and the oligosaccharide are indirectly linked via a linker or the like.

There is no particular restriction on the oligosaccharide that selectively captures a specific virus or bacterium (oligosaccharide that selectively captures a detection target) in the modified nanoparticle according to the present disclosure, insofar as the oligosaccharide selectively captures a specific virus or bacterium that is a detection target. Since the kind of the oligosaccharide, namely the sequence and/or the number of sugar residues constituting the oligosaccharide is uniquely tailored for the target virus or bacterium, an oligosaccharide with higher affinity for the virus or bacterium to be detected is appropriately selected.

As described above, the oligosaccharide preferably has high binding ability to the bacterium or virus.

The length of the oligosaccharide can be adjusted by the number of sugar residues in the oligosaccharide. Although there is no particular restriction on the number of sugar residues, the number of sugar residues may be, for example, from 2 to 10, or from 3 to 5.

The oligosaccharide may be either a naturally occurring oligosaccharide or an oligosaccharide that is not naturally occurring. The oligosaccharide may include a part that is modified.

Examples of the oligosaccharide include an N-linked sugar chain of a glycoprotein, an O-linked sugar chain of a glycoprotein, a polysaccharide, and a cyclodextrin. Among these, an oligosaccharide including sialic acid is preferable as an oligosaccharide for virus detection. Examples of the oligosaccharide including sialic acid include: α2,6-sialyl-N-acetyllactosamine (Neu5Ac(α2,6)Gal(β1,4)GlcNAc), α2,6-sialyllactosamine (Neu5Ac(α2,6)Gal(β1,4)GlcN), or α2,6-sialyllactose (Neu5Ac(α2,6)Gal(β1,4)Glc), which captures type A influenza virus; α2,3-sialyl-N-acetyllactosamine (Neu5Ac(α2,3)Gal(β1,4)GlcNAc), α2,3-sialyllactosamine (Neu5Ac(α2,3)Gal(β1,4)GlcN), or α2,3-sialyllactose (Neu5Ac(α2,3)Gal(β1,4)Glc), which captures avian influenza virus; sialyl 2,6-N-acetylgalactosamine (Neu5,9Ac₂(α2,6)GalNAc), sialyl 2,6-galactosamine (Neu5,9Ac₂(α2,6)GalN), or sialyl 2,6-galactose (Neu5,9Ac₂(α2,6)Gal), which captures human coronavirus; sialyl 2,3-N-acetylgalactosamine (Neu5,9Ac₂(α2,3)GalNAc), sialyl 2,3-galactosamine (Neu5,9Ac₂(α2,3)GalN), or sialyl 2,3-galactose (Neu5,9Ac₂(α2,3)Gal), which captures bovine coronavirus; a sialic acid residue (Neu4,5Ac₂), which captures mouse hepatitis virus; and a ganglioside (GD1a)(Neu4Ac(α2,3)Gal(β1,3)GalNAc(β1,4)Neu4Ac(α2,3)Gal(β1,4)Glc-OH), which captures adenovirus.

The oligosaccharide may be an oligosaccharide that selectively captures an influenza virus, particularly a specific type of influenza virus (a specific influenza virus that infects a specific species such as a human or a bird).

As examples, α2,6-sialyllactose (Neu5Ac(α2,6)Gal(β1,4)Glc), which captures type A influenza virus, and α2,3-sialyllactose (Neu5Ac(α2,3)Gal(β1,4)Glc), which captures avian influenza virus, will be described.

The structure of α2,6-sialyllactose (Neu5Ac(α2,6)Gal(β1,4)Glc) is shown below. Hemagglutinin on a human influenza virus recognizes the moiety, Neu5Ac(α2,6)Gal, in this sugar chain. That is, the structure in the dashed frame in the following chemical formula is a structure that a human influenza virus specifically recognizes. Further, α2,6-sialyl-N-acetyllactosamine (Neu5Ac(α2,6)Gal(β1,4)GlcNAc) also captures a human influenza virus. In addition, a sugar chain other than the above sugar chains may be used as a sugar chain that captures a human influenza virus, insofar as the sugar chain includes the Neu5Ac(α2,6)Gal moiety.

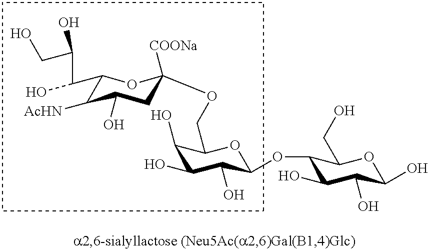

The structure of α2,3-sialyllactose (Neu5Ac(α2,3)Gal(β1,4)Glc) is shown below. Hemagglutinin on an avian influenza virus recognizes the moiety, Neu5Ac(α2,3)Gal. That is, the structure in the dashed frame in the following chemical formula is a structure that an avian influenza virus specifically recognizes. Further, α2,3-sialyl-N-acetyllactosamine (Neu5Ac(α2,3)Gal(β1,4)GlcNAc) also captures an avian influenza virus. In addition, a sugar chain other than the above sugar chains may be used as a sugar chain that captures an avian influenza virus, insofar as the sugar chain includes the Neu5Ac(α2,3)Gal moiety.

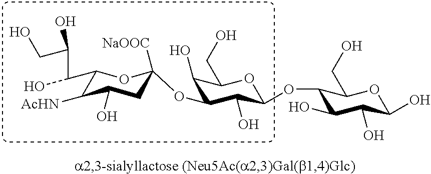

In this regard, Gal, Neu, Glc, and GalNAc represent kinds of sugar residues, namely Gal represents a galactose residue, Neu represents a N-acetylneuramic acid residue which is a sialic acid residue, Glc represents a glucose residue, and GalNAc represents a N-acetylgalactosamine residue. In addition, the notation between the respective sugar residues indicates a binding mode and a binding position. For example, Neu4Ac(α2,3)Glc represents that the position 2 of Neu4Ac and the position 3 of Glc are linked through an α-glycosidic bond. Neu4,5Ac₂ indicates that an acetyl group is bonded to each of positions 4 and 5 of a N-acetylneuramic acid residue.

At the nanoparticle surface side (the side linked to the nanoparticle surface) of the oligosaccharide such as those exemplified above, an additional sugar residue may exist. Even when the additional sugar residue is present, since a sugar chain that captures a detection target is present on the surface of a modified nanoparticle (at a side at which the oligosaccharide faces the medium around the modified nanoparticle), the modified nanoparticle is capable of attaching to the detection target.

The above oligosaccharide may be prepared from a natural product by a publicly known method, or may be prepared chemically or enzymatically by a publicly known method. Alternatively, a commercially available product may be used as it is, or the oligosaccharide may be prepared by chemical or enzymatic derivatization of a commercially available product. Examples of a commercially available oligosaccharides include α2,3-sialyl-N-acetyllactosamine, α2,3-sialyllactose, α2,6-sialyl-N-acetyllactosamine, and α2,6-sialyllactose.

The oligosaccharide may be directly bonded to the surface of a nanoparticle, or may be bonded through a linker. When the material of the nanoparticle is not suitable for direct bonding with the oligosaccharide, use of a linker is particularly useful.

When the oligosaccharide is bonded to the nanoparticle or the linker, there is no particular restriction on the bonding position in the oligosaccharide, insofar as the effect according to the present disclosure is obtained, and the nanoparticle or linker may be bonded to any position in sugar residues constituting the oligosaccharide. However, from the viewpoint of ease of bonding with the nanoparticle or linker, the bonding is preferably a bonding between a terminal carbon of the oligosaccharide having a reducible hemiacetal structure, and the nanoparticle or linker.

For example, when the surface of the nanoparticle (for example, a polystyrene nanoparticle) is modified with an amino group, it is possible to link the surface of the nanoparticle with the oligosaccharide using a compound having a carboxy group and a hydroxy group (such as glycolic acid). The amino group on the surface of the nanoparticle reacts with the carboxy group of the aforementioned compound to form an amide bond, and a hydroxy group of the oligosaccharide reacts with the hydroxy group of the aforementioned compound to form a glycosidic bond. When the surface of the nanoparticle (for example, a polystyrene nanoparticle) is modified with a carboxy group, it is possible to link the surface of the nanoparticle with the oligosaccharide using a compound having plural hydroxy groups, or having an amino group and a hydroxy group (such as ethylene glycol or ethanolamine). A carboxy group on the surface of the nanoparticle and the hydroxy or amino group of the aforementioned compound react to form an ester bond or an amide bond, and a hydroxy group of the oligosaccharide and the hydroxy group of the aforementioned compound react to form a glycosidic bond.

Further, when the surface of a nanoparticle (for example, a metal nanoparticle such as a gold nanoparticle) and an oligosaccharide are linked with a linker, it is preferable to form a linker using a linking compound that is a thiol-containing compound, and that has a functional group in addition to the thiol group. The thiol group may be a thiol group derived from a disulfide group. The presence of two or more different kinds of functional groups in the linking compound makes it easier to link the surface of the nanoparticle and the oligosaccharide. In the present disclosure, bond between a thiol and, for example, a metal nanoparticle can be easily attained by bringing the metal nanoparticle into contact with a solution containing a thiol group-containing compound (for example, by adding the metal nanoparticle into the solution). The reaction time for bonding may be, for example, from 20 min to 20 hours, or from 2 hours to 15 hours, and the reaction temperature may be, for example, from 5° C. to 40° C., or may be room temperature.

Examples of the functional group include an oxylamino group, a hydrazide group, an amino group, a hydroxy group, a carboxyl group, a carbonyl group, an azide group, an alkynyl group, an epoxy group, and an isocyanate group, in addition to the aforedescribed thiol group. Furthermore, the functional group other than a thiol group may be an oxylamino terminal or a hydrazide terminal in consideration of the ability to bind to a carbon at a reducing terminal of the oligosaccharide. When the terminal at a side for bonding to the oligosaccharide is an oxylamino group or a hydrazide terminal, it is not necessary to provide the oligosaccharide with a functional group for bonding, and the oligosaccharide can be used, as it is, for bonding to the linking compound.

As the linking compound, for example, a single compound having a thiol group together with a functional group other than a thiol group or a functional group other than a disulfide group may be used, or a multiple compounds each having a thiol group together with a functional group other than a thiol group or a functional group other than a disulfide group may be used.

The linker that links the oligosaccharide with the surface of the nanoparticle may be, for example, a linker represented by -P¹-T¹-X¹-, in which P¹ is —S—, —COO—, —CONH—, —NHCO—, or —OCO—. T¹ is a hydrocarbon linking group having from 1 to 20 carbon atoms that may contain one or two ester or amide bonds (of which direction is not limited). X¹ represents a single bond or a linking group to an oligosaccharide. The hydrocarbon linking group represented by T¹ is preferably a straight-chain alkylene group having from 1 to 15 carbon atoms, a straight-chain alkenylene group having from 2 to 15 carbon atoms, a branched alkylene group having from 3 to 15 carbon atoms, a branched alkenylene group having from 4 to 15 carbon atoms, a cyclic alkylene group having from 6 to 15 carbon atoms, or an arylene group having from 6 to 15 carbon atoms, each of which may contain one or two ester or amide bonds, or —CH₂13 CH₂—(O—CH₂—CH₂)ₙ— (n is 0 or a freely-selected natural number, preferably an integer from 0 to 20, and more preferably an integer from 0 to 10). The linking group represented by X¹, which is bound to the oligosaccharide, is preferably —O—N═ or —NH—N═. A valence electron of P¹ forms a bond to the surface of the nanoparticle. When X¹ is a single bond, T¹ is bonded to the oxygen at the reducing terminal of the oligosaccharide, and when X¹ is —O—N═ or —NH—N═, X¹ is bonded to the carbon of the aldehyde moiety of the reducing terminal (ring-opened) of the oligosaccharide to form an oxime.

When the surface of the nanoparticle (for example, a metal nanoparticle such as a gold nanoparticle) and the oligosaccharide are linked by a linker, the linking may be achieved by, for example, linking the reducing terminal of the oligosaccharide to a compound having a hydroxy group and an amino group (for example, ethanolamine) by a dehydration reaction (attachment of an amino group-containing structure), and, separately, allowing a compound having a thiol group and a carboxy group (for example, 11-mercaptoundecanoic acid) to react with the surface of the nanoparticle to bond the thiol group to the surface of the nanoparticle, and then allowing the amino group attached to the oligosaccharide and the carboxy group attached to the nanoparticle to react with each other to be mutually linked by an amide bond. Examples of the compound to be reacted with the reducing terminal of the oligosaccharide include methanolamine and propanolamine, in addition to ethanolamine. When these compounds are allowed to react, 2-aminoethyl, aminomethyl, or 3-aminopropyl, respectively, will be bonded to the oxygen atom of the reducing terminal. In other words, ethylamine, methylamine, propylamine, or the like can be used as a linking group that connects the oligosaccharide (for example, α2,3-sialyl-N-acetyllactosamine or α2,6-sialyl-N-acetyllactosamine) and a nanoparticle-side portion of the linker (or as a part of T¹). Examples of the compound to be reacted with the surface of the nanoparticle include 8-mercaptoheptanoic acid and 12-mercaptododecanoic acid, in addition to 11-mercaptoundecanoic acid. It is known that a thiol group has high bonding ability with respect to, particularly, a metal, and is favorably used for bonding to the surface of a metal nanoparticle. In particular, when a thiol group is used on the surface of a gold nanoparticle, the surface of the gold nanoparticle can easily be modified with various molecules through an S—Au bond. When the aforementioned reaction between the amino group and the carboxy group is performed, the reaction may be promoted by allowing a condensing agent, such as 4-(4,6-dimethoxy-1,3,5-triazin-2-yl)-4-methylmorpholinium chloride n-hydrate (DMT-MM), to be present. Surplus free oligosaccharides and other by-products remaining after the reaction, can be removed by dialysis with a dialysis membrane (for example, a dialysis membrane with a cutoff of 3.5 kDa).

In the present disclosure, a dehydration reaction between a hydroxy group at the reducing terminal of the oligosaccharide and an alcohol (for example, ethanolamine) can be advanced, for example, by reacting the oligosaccharide and the alcohol in the presence of an acid catalyst, and the dehydration reaction may be performed under reduced pressure. For example, the reaction may be performed by adding the alcohol in an excessive amount relative to the hydroxy group at the reducing terminal of the oligosaccharide, at from about 60° C. to about 100° C. for from about 0.5 to about 40 hours. Examples of the acid catalyst include hydrochloric acid, sulfuric acid, phosphoric acid, and p-toluenesulfonic acid. Hydroxy groups in the oligosaccharide other than the hydroxy group at the reducing terminal may be protected with a protecting group, as appropriate.

In the present disclosure, formation of an amide bond by dehydration condensation between an amino group and a carboxy group may be performed under an acidic condition while heating. The formation of an amide bond may be performed by once converting the carboxy group into an acid chloride or an acid anhydride, and then allowing the acid chloride or acid anhydride to react with an amino group. Examples of such a reaction include the Schotten-Baumann reaction in which an acid chloride and an amino group are allowed to react with each other in water or a water-containing solvent in the presence of sodium hydroxide or sodium carbonate. However, from the viewpoint of performing quantitative dehydration condensation under a mild condition close to neutrality, it is preferable to perform dehydration condensation using a condensing agent. Examples of the condensing agent include N′,N′-dicyclohexylcarbodiimide (DCC), water-soluble carbodiimide (WSCD), carbonyldiimidazole (CDI), 1-hydroxybenzotriazole (HOBt), 1-hydroxy-7-azabenzotriazole (HOAt), diphenyl phosphate azide (DPPA), a BOP reagent, o-(benzotriazol-1-yl)-N,N,N′,N″-tetramethyluronium hexafluorophosphate (HBTU), HATU, TATU, TBTU, 2-chloro-4,6-dimethoxy-1,3,5-triazine (CDMT), and 4-(4,6-dimethoxy-1,3,5-triazin-2-yl)-4-methylmorpholinium chloride n-hydrate (DMT-MM). When a condensing agent is used, the reaction may be performed, for example, under conditions of from 0° C. to 50° C., or from 10° C. to 35° C., for from 0.5 hours to 30 hours, or from 1 hour to 20 hours. The pH may be, for example, from 4 to 10, or from 5 to 9.

In the present disclosure, formation of an ester bond by dehydration condensation between a hydroxy group and a carboxy group may be performed under an acidic condition while heating. The formation of an ester bond may be performed by once converting the carboxy group into an acid chloride or an acid anhydride, and then allowing the acid chloride or acid anhydride to react with a hydroxy group. Examples of such a reaction include a Fischer esterification reaction. However, from the viewpoint of performing quantitative dehydration condensation under a mild condition close to neutrality, it is preferable to perform dehydration condensation using a condensing agent. Examples of the condensing agent include N′,N′-dicyclohexylcarbodiimide (DCC), carbonyldiimidazole (CDI), 2,4,4-trichlorobenzoyl chloride, 2-methyl-6-nitrobenzoic anhydride, and dimesitylammonium pentafluorobenzenesulfonate. The condensing agents mentioned as examples of the condensing agent for formation of an amide bond can also be used for formation of an ester bond, insofar as its ability to activate a carboxy group is sufficient to cause an ester formation reaction. When a condensing agent is used, the reaction may be performed, for example, under conditions of from 0° C. to 50° C., or from 10° C. to 35° C., for from 0.5 hours to 30 hours, or from 1 hour to 20 hours. The pH may be, for example, from 4 to 10, or from 5 to 9.

As examples in which an amino group-containing structure is linked to the reducing terminal of the oligosaccharide, an example in which a 2-aminoethyl group is linked to α2,6-sialyl-N-acetyllactosamine (α2,6-sialyl-N-acetyllacsamine-β-ethylamine), and an example in which a 2-aminoethyl group is linked to α2,3-sialyl-N-acetyllactosamine (α2,3-sialyl-N-acetyllacsamine-β-ethylamine) are shown below.

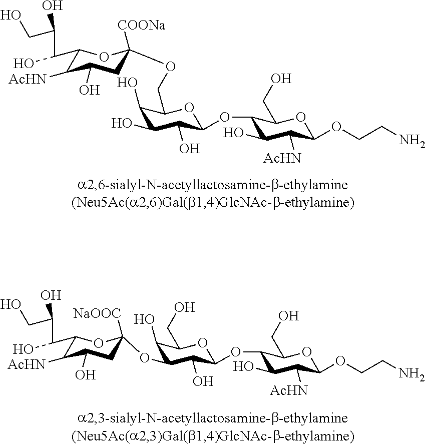

In an embodiment, the nanoparticle or the linker has an oxylamino terminal. In this case, the hemiacetal at the reducing terminal of the oligosaccharide easily becomes an aldehyde in reducing conditions, and the resultant aldehyde reacts with an oxylamino group at the nanoparticle surface or at the linker, thereby enabling formation of a stable oxime structure.

In addition, the above oxylamino group bonds to an oligosaccharide more easily than other functional groups do, and an oxime, which is stable in an aqueous solution, is formed by this bonding. For this reason, when the nanoparticle surface or the linker has an oxylamino group and another functional group, it is possible to cause only the bonding between the oligosaccharide and an oxylamino group while preventing the oligosaccharide from bonding to another functional group. Therefore it is possible to introduce a substituent other than the oligosaccharide to the other functional group at the nanoparticle surface or at the linker.

Although there is no particular restriction on the mode of binding of the oligosaccharide to the nanoparticle surface or the linker insofar as an effect according to the present disclosure can be obtained, it is possible to introduce the oligosaccharide (such as α2,3-sialyllactose or α2,6-sialyllactose), for example, to a functional group present at the nanoparticle surface or at the linker using a “glycoblotting” technique described in International Publication No. WO 2004/058687. In this case, for example, the reaction conditions for allowing the oligosaccharide and an oxylamino group at the nanoparticle surface or at the linker to react with each other preferably include reaction at from 50° C. to 70° C. for from 140 min to 240 min. When using the glycoblotting technique, a commercially available kit such as a kit (BlotGlyco) produced by Sumitomo Bakelite Co., Ltd. can be used.

There is no particular restriction on the virus or bacterium that is the detection target in the present disclosure, insofar as the virus or bacterium can be captured by an oligosaccharide. Examples of bacteria that can be captured by an oligosaccharide include bacteria which have pathogenicity and possibility of being captured by an oligosaccharide, such as Mycoplasma, Mycobacterium tuberculosis, Streptococcus, Bordetella pertussis, Legionella, Pseudomonas aeruginosa, various pathogenic Escherichia coli species, Clostridium perfringens, Clostridium tetani, Clostridium difficile, Helicobacter pylori, Shigella, and Neisseria meningitides. Also, some lactic acid bacteria (including Bifidobacterium) and non-pathogenic bacteria can be selected as a detection target insofar as such bacteria can be captured by an oligosaccharide.

Further, examples of viruses that can be captured by an oligosaccharide include influenza viruses (type A (including subspecies), type B, and type C), parainfluenza virus, norovirus, adenovirus, dengue virus, herpes virus, coronavirus, rhinovirus, and mouse hepatitis virus (MHV).

The amount of the oligosaccharide, which selectively captures the detection target, on the nanoparticle is preferably 10% or more, more preferably 30% or more, and further preferably 50% or more, in terms of the ratio (coverage) with respect to the maximum number of oligosaccharide molecules that can be bound onto the nanoparticle (namely, the number of oligosaccharide molecules bound onto the nanoparticle when the binding is saturated). There is a tendency that an increased number of bound oligosaccharide molecules provides enhanced attachment capability of the modified nanoparticle to the detection target. However, since dispersibility improving groups are also to be bonded to the nanoparticle, an excessively high coverage by the oligosaccharide may reduce the number of dispersibility improving groups that can be bound to the nanoparticle, and may reduce the dispersibility improving effect. From this point of view, the coverage by the oligosaccharide is preferably 95% or less, and more preferably 90% or less.

### Dispersibility Improving Group

In the modified nanoparticle according to the present disclosure, a dispersibility improving group is bonded to the nanoparticle surface. In the present disclosure, the bond between the dispersibility improving group and the nanoparticle surface is not limited to a mode in which the dispersibility improving group and the nanoparticle surface are directly bonded, but a mode in which the dispersibility improving group and the nanoparticle surface are indirectly linked via a linker or the like is also contemplated.

The dispersibility improving group in the modified nanoparticle according to the present disclosure may be any group that improves the dispersibility of the nanoparticle in a solvent, the nanoparticle having the oligosaccharide, which selectively captures a specific virus or bacterium, bonded to the surface of the nanoparticle. When the dispersibility improving group is bound to the surface of the nanoparticle, not only the dispersibility of the modified nanoparticles is improved, but also a surprising effect that the selectivity of attachment to the detection target is also improved is obtained. The dispersibility improving group is, for example, a group having a hydrophilic moiety (for example, an amino group or a carboxy group) that improves dispersibility in a hydrophilic solvent, and the dispersibility improving group may be attached to the nanoparticle through a moiety that bonds to the nanoparticle (for example, a thio structure).

The dispersibility improving group may be a betaine structure-containing group. When a betaine structure-containing group is used, aggregation and precipitation due to hydrophobic interaction between modified nanoparticles is reduced, and dispersibility improves. As a result, non-specific attachment of the modified nanoparticle to a structure that is not a detection target can be further effectively reduced. This is presumably because a firm hydrated surface is formed when a betaine structure-containing group is bonded onto the nanoparticle.

Examples of the betaine structure include: a sulfobetaine group, which includes an amino group and a sulfo group; a carboxybetaine group, which includes an amino group and a carboxy group; and a phosphobetaine group, which includes an amino group and a phosphate group. Examples thereof include the structure of Formula A illustrated below. Examples of a betaine structure-containing group include the following sulfobetaine-3-undecanethio group. Although use of (methacryloyloxy)phosphatidylcholine or polyethylene glycol is also contemplated, an excessively large dispersibility improving group may hinder the oligosaccharide from accessing the detection target in some cases.

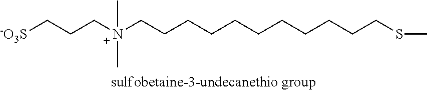

A betaine structure-containing group can be fixed onto a nanoparticle by binding a compound having the betaine structure (hereinafter also referred to as a “dispersibility improver having a betaine structure”) onto the nanoparticle. The betaine structure-containing group may be bound to the surface of the nanoparticle through, for example, a thio group. For example, by allowing a compound having a betaine structure-containing group and a thiol group (as a dispersibility improver) to react with the surface of the nanoparticle, the betaine structure-containing group can be linked to the surface of the nanoparticle through a thio group. For example, binding of the afore-described sulfobetaine-3-undecanethio group to the surface of a gold nanoparticle can be accomplished by binding N-(11-mercaptoundecyl)-N,N-dimethyl-3-ammonio-1-propanesulfonate (SB-SH) (also referred to as “sulfobetaine-3-undecanethiol”) to the surface of the gold nanoparticle.

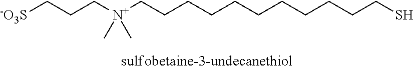

The dispersibility improving group may be, for example, a group represented by -P²-T²-X², in which P² is —S—, —COO—, —CONH—, —NHCO—, or —OCO—, T² is a hydrocarbon linking group having from 1 to 15 carbon atoms, and X² represents a betaine group. The hydrocarbon linking group represented by T² is preferably a straight-chain alkylene group having from 1 to 15 carbon atoms, a straight-chain alkenylene group having from 2 to 15 carbon atoms, a branched alkylene group having from 3 to 15 carbon atoms, a branched alkenylene group having from 4 to 15 carbon atoms, a cyclic alkylene group having from 6 to 15 carbon atoms, an arylene group having from 6 to 15 carbon atoms, or —CH₂—CH₂—(O—CH₂—CH₂)ₘ— (wherein m is 0 or any freely-selected natural number, preferably an integer from 0 to 20, and more preferably an integer from 0 to 10). The betaine group represented by X² is preferably —N⁺(R¹)(R²)-Y-Z (see the following formula A). In Formula A, * represents a connection point to T².

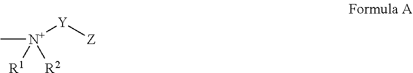

In Formula A, R¹ and R² are independently a straight-chain alkylene group having from 1 to 8 carbon atoms, a straight-chain alkenylene group having from 2 to 8 carbon atoms, a branched alkylene group having from 3 to 8 carbon atoms, a branched alkenylene group having from 4 to 8 carbon atoms, a cyclic alkylene group having from 6 to 8 carbon atoms, or an arylene group having from 6 to 8 carbon atoms; Y is a single bond, a straight-chain alkylene group having from 1 to 8 carbon atoms, a straight-chain alkenylene group having from 2 to 8 carbon atoms, a branched alkylene group having from 3 to 8 carbon atoms, a branched alkenylene group having from 4 to 8 carbon atoms, a cyclic alkylene group having from 6 to 8 carbon atoms, or an arylene group having from 6 to 8 carbon atoms; and Z represents —SO₃⁻, —COOH, or —PO₃⁻. The valence electron of P² forms a bond to the surface of a nanoparticle.

The amount of dispersibility improving groups on the nanoparticle is preferably 10% or more, more preferably 30% or more, and further preferably 50% or more, in terms of the ratio (coverage) with respect to the maximum number of dispersibility improving groups that can be bound onto the nanoparticle (namely, the number of dispersibility improving groups bound onto the nanoparticle when the binding is saturated). There is a tendency that an increased number of bound dispersibility improving groups provides enhanced dispersibility of the modified nanoparticle. However, since the oligosaccharide that selectively captures the detection target is also to be bound to the nanoparticle, an excessively high coverage by dispersibility improving groups may reduce the number of oligosaccharide molecules that can be bound to the nanoparticle, and may reduce the selectivity for bonding to the detection target. From this point of view, the coverage by dispersibility improving groups is preferably 80% or less, and more preferably 60% or less.

The modified nanoparticle according to the present disclosure may be prepared by performing a reaction of binding an oligosaccharide, that selectively captures a detection target, onto a nanoparticle, and a reaction of binding a dispersibility improving group onto the nanoparticle. Among these reactions, the reaction of binding the oligosaccharide onto the nanoparticle may be performed before the other reaction, or the reaction of binding a dispersibility improving group onto the nanoparticle may be performed before the other reaction, or both reactions may be performed at the same time. In the case of preparation of the aforementioned metal nanoparticle, the preparation preferably includes allowing a compound having a thiol group and a carboxy group (for example, 11-mercaptoundecanoic acid) to react with the surface of a nanoparticle, and, at the same time, also allowing a compound having a betaine structure-containing group and a thiol group to react with the surface of the nanoparticle, and thereafter allowing an amino group attached to the oligosaccharide and a carboxy group attached to the nanoparticle to react with each other to form a linkage with an amide bond. According to the preparation in this manner, a situation in which a structure that is either the oligosaccharide or the dispersibility improving group and that is linked to the nanoparticle first hinders the subsequent reaction for linking the other structure to the nanoparticle can be avoided. The molar ratio of the compound having a thiol group and a carboxy group to the compound having a betaine structure-containing group and a thiol group may be from 2:8 to 8:2, or from 4:6 to 6:4. The compound having a thiol group and a carboxy group and the compound having a betaine structure-containing group and a thiol group may be used in equimolar amounts.

The reaction of binding an oligosaccharide that selectively captures a detection target on to a nanoparticle, or the reaction of binding a dispersibility improving group onto a nanoparticle can be performed by dispersing nanoparticles in an appropriate solvent, and allowing substances for use in the reaction to be co-present in the dispersion liquid. The reaction conditions (pH, temperature, salt concentration, etc.) during the reaction may be selected according to ordinary methods.

The overall particle size of the modified nanoparticle (a size including the nanoparticle as well as the oligosaccharide and the dispersibility improving groups bound to the nanoparticle surface) can be measured by dynamic light scattering (DLS), for example, using a particle size measurement device (ZETASIZER NANO ZS (trade name) of Malvern Panalytical). The average particle size (volume average particle size as measured by DLS) may be, for example, in a range of from 10 nm to 220 nm, or in a range of from 15 nm to 120 nm, or in a range of from 20 nm to 120 nm, or in a range of from 30 nm to 70 nm.

When the modified nanoparticle according to the present disclosure has been mixed, for example, with a sample collected from a living body, the modified nanoparticle attaches to the detection target, if any, present in the sample. This attachment can be detected by a particle size analysis method. Therefore, by using the modified nanoparticle according to the present disclosure, it is possible to detect the presence or absence of the detection target in the sample. Examples of a method used for analyzing the particle size include resistive pulse sensing, which will be described below, dynamic light scattering, measurement with a transmission electron microscope (TEM), and impedance measurement. Resistive pulse sensing is preferable because resistive pulse sensing enables rapid measurement and obtainment of a particle size distribution. The method used for detecting the attachment of the modified nanoparticle is not limited those observing a particle size change (shift), and the detection may be performed by any method in which the nanoparticle itself or a structure (such as a fluorescent chromophore) that serves as a label and that is bonded to the nanoparticle.

### Dispersion Liquid That Contains Modified Nanoparticle and Aqueous Medium

A dispersion liquid according to the present disclosure that contains a modified nanoparticle and an aqueous medium is a dispersion liquid that contains an aqueous medium and the modified nanoparticle according to the present disclosure dispersed in the aqueous medium. In the dispersion liquid, the modified nanoparticle can freely move around, and, if the detection target is present, the modified nanoparticle can attach to the detection target. For example, when the dispersion liquid has been mixed with a sample collected from a living body, the modified nanoparticle attaches to the detection target, if any, present in the sample. This attachment can be detected by the above exemplary techniques described in the description of the modified nanoparticle. Further, since modified nanoparticle according to the present disclosure has high dispersion stability due to the presence of a dispersibility improving group, the dispersion liquid according to the present disclosure can be stored in a stable state over a long period of time.

There is no particular restriction on the aqueous medium used in the dispersion liquid according to the present disclosure, insofar as the aqueous medium is water, a water-soluble organic solvent, or a mixed liquid of water and a water-soluble organic solvent. Examples of the water-soluble organic solvent include an alcohol, such as methanol or ethanol, and a glycol, such as diethylene glycol or polyethylene glycol. The aqueous medium may contain a buffer substance such as Tris-HCl or PBS (for example, ⅓×PBS). The pH of the aqueous medium is preferably at a level at which the performance of the oligosaccharide that selectively captures the detection target and the performance of the dispersibility improving group are not significantly reduced, and, specifically, the pH may be from 5 to 9, or from 6 to 8.

The dispersion liquid according to the present disclosure can be obtained by dispersing the modified nanoparticle according to the present disclosure in an aqueous medium. The dispersing may be performed using a stirring instrument or a stirring apparatus, such as a stirrer, a paddle mixer, an impeller mixer, a homomixer, a disperser mixer, or an ultramixer.

### Set for Resistive Pulse Sensing Including Modified Nanoparticle or Dispersion Liquid and Pore-Containing Membrane for Resistive Pulse Sensing

A set for resistive pulse sensing according to the present disclosure includes the modified nanoparticle or dispersion liquid according to the present disclosure and a pore-containing membrane for resistive pulse sensing. As described below, resistive pulse sensing is a technique in which the particle size or the like of a particle is measured by providing the first chamber and the second chamber separated by a membrane as boundary, applying a voltage between the first chamber and the second chamber, and detecting an increase in electrical resistance that occurs when a particle in a sample introduced into the first chamber passes through a minute pore provided in the membrane in the course of migrating to the second chamber.

Since the set for resistive pulse sensing according to the present disclosure includes the modified nanoparticle or dispersion liquid according to the present disclosure and a pore-containing membrane for resistive pulse sensing, high-sensitivity selective detection of the detection target in a sample is possible when the set is mounted on a resistive pulse sensing device installed in a medical facility or the like. Details of the pore-containing membrane for resistive pulse sensing will be described below.

### Set for Detection of Specific Virus or Bacterium

A set for detection of a specific virus or bacterium according to the present disclosure includes modified nanoparticles or a dispersion liquid according to the present disclosure, and a resistive pulse sensing device. Since the set for detection of a specific virus or bacterium according to the present disclosure includes the modified nanoparticle or dispersion liquid according to the present disclosure and a resistive pulse sensing device, high-sensitivity selective detection of the detection target is enabled by mixing a sample such as a biological sample and the modified nanoparticle or dispersion liquid according to the present disclosure, and performing a measurement using a resistive pulse sensing device. Details of the resistive pulse sensing device will be described below.

### Reagent for Detection of Specific Virus or Bacterium

A reagent for detection of a specific virus or bacterium by resistive pulse sensing according to the present disclosure (hereinafter also referred to as the “reagent according to the present disclosure”) includes the modified nanoparticle according to the present disclosure. The reagent according to the present disclosure may be the modified nanoparticle according to the present disclosure itself, or may further contain a dispersion medium such as water or a buffer solution.

When the reagent for detection of a specific virus or bacterium according to the present disclosure is used, the oligosaccharide that selectively captures a specific virus or bacterium specifically captures the detection target, whereby the modified nanoparticle selectively attaches to the detection target. The attachment of the modified nanoparticles can be detected using resistive pulse sensing measurement, by detecting a size of the modified nanoparticles-detection target complex that is increased as compared to the size of the detection target alone. By detecting the attachment of the modified nanoparticles, the presence and/or amount of the detection target can be measured.

The reagent can be used to detect the specific virus or bacterium based on, for example, the presence or absence of a shift of a particle size peak in the particle size distribution.

Heretofore, no attempts have been made to detect a detection target by resistive pulse sensing using a nanoparticle having a surface-bound oligosaccharide that selectively captures a specific virus or bacterium. For example, a method that has been widely used for detecting an influenza virus is immunochromatography. However, the importance of detection at an early stage of infection, at which administration exerts a significant effect, is reaffirmed with the development of antiviral drugs, and the detection sensitivity attainable by immunochromatography still has room for improvement.

Since elderly people tend to get worse when they are infected with an infectious disease, the elderly population is exposed to a threat of serious infectious diseases such as influenza. For example, influenza infection often causes infectious complications such as pneumonia, with serious consequences. Since the elderly people often have a weakened immune system, there is a desire for improved diagnostic techniques to enable detection at an early stage. Currently, parallel flow immunochromatography is widely used as a diagnostic method. However, even with this technique, a target disease cannot always be detected due to low detection sensitivity. Since most drugs against an influenza virus are a neuraminidase inhibitor and should be administered within 48 hours of infection, improvement of the detection sensitivity is one of the most important issues to be solved.

In the case of immunochromatography, only a small amount of information can be obtained from individual virus particles, and only the information about a group can be obtained after a certain number of particles are gathered. In contrast, resistive pulse sensing used in the present disclosure obtains information about individual particles in the form of changes in electrical resistance, and the content of information is not limited to detection of the presence of individual particles but also encompasses obtainment of information concerning the sizes of individual particles. Thus, detection using resistive pulse sensing is possible even when the number of virus particles is smaller than a number at which detection by immunochromatography is possible.

However, conventional measurement of virus particles by resistive pulse sensing has not been able to distinguish virus particles having similar sizes. This is because such virus particles give peaks at similar positions in terms of resistance peak signals. For this reason, it has been difficult to distinguish between specific types of influenza viruses, such as human influenza virus and avian influenza virus.

Specifically, influenza viruses have a size of from 80 to 120 nm, but there are several types and subtypes, and, therefore, the characteristics of influenza viruses are diverse. A highly pathogenic avian influenza (HPAI) in a human is known as a newly occurring infectious disease with a higher mortality rate compared to a human influenza virus. However, it was difficult to distinguish between a human influenza virus and an avian influenza virus using the difference in physical properties between the two influenza A viruses.

In contrast, in the present disclosure, a nanoparticle (modified nanoparticle) is used which has a surface-bound oligosaccharide that selectively captures a detection target and a surface-bound dispersibility improving group, and attachment of the modified nanoparticle to the detection target is detected by resistive pulse sensing as a particle size change of the detection target (difference between the particle size of the detection target itself and the particle size of a detection target-modified nanoparticles complex). Owing to this configuration, different types or subtypes of influenza viruses with similar particle sizes can also be distinguished from each other based on the capture selectivity of the oligosaccharide. As a result, it is possible to detect a specific kind (type, subtype, or the like) of influenza virus with high sensitivity.

There was an example in which detection of a detection target using a nanoparticle was carried out by using a nanoparticle on which an antibody was immobilized. However, preparation of an antibody is time- and labor-consuming, and since an antibody is composed of polypeptides, it is sometimes difficult to maintain its stability for a long period of time. Furthermore, since the site in an antigen that is recognized by an antibody cannot be designed in advance, even an antibody that binds to a virus is not necessarily able to distinguish between similar kinds of viruses (type, subtype, or the like). In the present disclosure, these problems associated with the use of an antibody are overcome by using an oligosaccharide that selectively captures a detection target for capturing the detection target, and also using a dispersibility improving group for reducing non-specific attachment.

Although an influenza virus as an example is used in the above explanations, the same explanations shall apply to other viruses and bacteria.

In the reagent according to the present disclosure, a dispersibility improving group is further bonded to the nanoparticle. Due to this configuration, non-specific attachment of the modified nanoparticle to co-existing substances and the like is reduced, and the selectivity in capturing the detection target by the modified nanoparticle is further improved.

### Method of Detecting Virus or Bacterium

A method of detecting a virus or bacterium according to the present disclosure includes:

(a) a step of measuring a particle size distribution of particles included in a biological liquid sample by resistive pulse sensing;

(b) a step of preparing a mixed liquid by mixing the biological liquid sample with the reagent according to the present disclosure; and (c) a step of measuring a particle size distribution of particles included in the mixed liquid by resistive pulse sensing;

wherein the biological liquid sample is judged to include the virus or bacterium when there is a peak in a particle size range corresponding to the virus or bacterium, of which a peak position in the particle size distribution obtained in the step (c) exhibits a shift toward a larger particle size side, compared to a peak position thereof in the particle size distribution obtained in the step (a).

### Resistive Pulse Sensing

Resistive pulse sensing is a technique in which a change in electrical resistance occurring when a particle passes through a pore is measured. Specifically, a resistive pulse sensing device includes the first chamber, the second chamber, and a membrane that is provided as a partition between the first and second chambers and that contains a minute pore. The first chamber and the second chamber are filled with an electrolytic solution. For measurement, a liquid sample is added to the first chamber, and a voltage is applied between the first chamber and the second chamber. The voltage can be applied, for example, by providing an electrode on each of a wall of the first chamber and a wall of the second chamber, and applying an electric potential difference between these electrodes. When the voltage is applied, electric current flows between the electrodes. When a particle passes through a pore that connects the first chamber and the second chamber, the electric current decreases (in other words, the resistance value increases) temporarily. According to the Maxwell's theory, the increase in the resistance is proportional to the volume of the electrolytic solution displaced by the particle (i.e. the volume of the particle). Therefore, the number of passing particles and the sizes of the respective particles can be measured by monitoring the changes in the electric resistance value. This is the principle of resistive pulse sensing.

In a signal (pulse) of an increase in electrical resistance (blockade of an ionic current), the height of the signal (i.e. the magnitude of the increase in resistance) represents the size of the passing particle, and the duration of the signal reflects the migration speed of the particle. Since the ion velocity of the particle is influenced not only by the pressure difference applied between the chambers but also by the voltage applied between the chambers, it is also possible to determine the zeta potential or the like of the particle based on the information on the duration.

Various salt solutions can be used as the electrolytic solution, and the electrolytic solution is preferably a physiological buffer solution. For example, a PBS buffer solution such as ⅓×PBS, a Tris buffer solution, or the like may be used. When the size of the pore is large, the electric current value will be large; therefore it is preferable to lower the molar concentration of the electrolyte.

In resistive pulse sensing, a flow passing through the pore may be created by providing a pressure difference between the first chamber and the second chamber. Although particles in a measurement sample spontaneously pass through the pore owing to the electric charges the particles themselves, induced passage through the pore due to the generated physical flow enables measurement of many particles in a shorter time period.

When the voltage applied between the electrodes is too low, the electric current will be weak and the measurement accuracy will be deteriorated. When the voltage is too high, the amount of the resultant ionic current may be too large, or a short circuit may occur. Insofar as these problems do not occur, there is no particular restriction on the voltage. The voltage may be, for example, from 10 mV to 100 V, or from 50 mV to 10 V. Further, the pressure difference between the chambers is not particularly limited, either, and the pressure difference may be, for example, from 0.005 kPa to 5 kPa, or from 0.01 kPa to 2.0 kPa.

Although there is no particular restriction on the volumes of the first and second chambers, the volumes of the first and second chambers each may be, for example, from 0.1 mL to 50 mL, or from 0.5 mL to 10 mL. The amount of the liquid sample to be added may be, for example, from 10 μL to 1 mL, or from 30 μL to 0.5 mL. The liquid sample to be added is preferably prepared such that the particle concentration in the liquid sample is in a range of from 10⁵ particles/mL to 10¹² particles/mL, from the viewpoint of quickly and accurately detecting the electric resistance value peaks generated by the respective particles.

Examples of the resistive pulse sensing device include qNANO (trade name) manufactured by Izon Science Ltd. In this regard, COULTER COUNTER series manufactured by Beckman have a lower limit of measurable particle diameter of about 400 nm, and cannot be used for measurement of a virus having a size of, for example, about 100 nm. However, COULTER COUNTER series may be used for measurement of a detection target having a size larger than the foregoing lower limit value.

The particle sizes of particles included in the liquid sample are preferably in a range of from 40 nm to 10 μm. When particles having excessively large particle sizes are included, the pore will be clogged and correct measurement is not possible. Therefore, a liquid sample that may possibly include coarse particles may be subjected to measurement after the coarse particles are removed by filtration with a filter (for example, a filter having openings of 500 μm or 100 μm), dialysis, or the like.

A membrane serving as a partition between the first and the second chambers (a pore-containing membrane for resistive pulse sensing) is, for example, a polymer membrane, and more preferably a polyurethane membrane. Although there is no particular restriction on the membrane thickness, the membrane thickness is, for example, from 0.1 mm to 5 mm, and the membrane thickness may be from 0.5 mm to 3 mm.

The pore diameter of the membrane may be selected in accordance with the sizes of particles that are expected to be contained in the liquid sample, within a range of, for example, from 40 nm to 10 μm. For example, as membranes for the aforementioned qNANO, pore-containing membranes for resistive pulse sensing having different pore sizes, such as NP-100, NP-150, NP-200, NP-300, NP-400, NP-800, NP-1000, NP-2000, and NP-4000 (all of which are trade names) are available from Izon Science Ltd. The shape of the membrane may be round, square, rectangle, or another polygonal shape. Alternatively, it is preferable to provide the membrane with four arms respectively extending in directions at an angle of 90 degrees therebetween for adjustment of the pore size, and to apply an appropriate tension between the arms, thereby enabling adjustment of the pore size to an appropriate size. In this case, the shape of the membrane is a cross shape. The aforementioned membranes available from Izon Science Ltd. have such variable pore sizes.

The resistive pulse sensing using such variable pore size is called tunable resistive pulse sensing (TRPS), and use of TRPS is preferable in terms of obtaining high measurement sensitivity in the present disclosure.

The shape of the pore in the cross section of the membrane may be a cylindrical shape, or a conical shape lacking an apical part, but a conical shape lacking an apical part is preferable. When the shape of the pore is a conical shape lacking an apical part, the peak appears more sharply, and it becomes easier to distinguish and separate peaks of individual particles.

For details of resistive pulse sensing, documents such as Nano Today, 2011 Oct. 1; 6(5): 531-545. doi: 10.1016/j.nantod.2011.08.012, which is incorporated by reference herein, may be referenced.

When a very small pore is to be used, a pore of a membrane transport protein can be utilized. However, an issue associated with detection using a membrane transport protein is limitation in size and degradation. Therefore, artificial nanopores or micropores have been developed, as described above, for detection of various particles, such as a nucleic acid, a peptide, a protein, a whole bacterium, a whole virus, or an extracellular vesicle. The size distribution of a polydisperse nanoparticle sample can be calculated quickly and accurately by resistive pulse sensing, similarly to the case of a TEM image. In contrast, it is not possible to determine the size of each individual particle by dynamic light scattering (DLS) when a polydisperse sample is measured.

Each kind of virus has its unique size. For example, type A influenza virus has a diameter of from 80 to 120 nm, and, in contrast, a picornavirus such as enterovirus has a diameter of 30 nm. The pore diameter is preferably a diameter that enables the detection target to pass through, and that is not excessively large as compared to the size of the detection target. For example, the maximum diameter of the detection target is preferably from 5% to 90% of the pore diameter, and more preferably from 10% to 85% of the pore diameter.

According to the present disclosure, contribute to early diagnosis for prevention of epidemic diseases can be made by distinguishing individual viruses such as influenza. Recently, a highly pathogenic avian influenza (HPAI) such as H5N1 influenza subspecies A is known as an epidemic disease with high mortality in humans. Subtypes of type A influenza virus have almost the same virus physical properties. These subtypes can also be separately detected according to the present disclosure.

In the present disclosure, resistive pulse sensing measurement is preferably carried out using qNANO (trade name) manufactured by Izon Science Ltd. on which a membrane for qNANO, such as NP-100, or NP-150 (trade name), is mounted. The setting for the measurement may be made according to the manufacturer's manual, and may be a default setting.

In the method of detecting a virus or bacterium according to the present disclosure, the particle size distribution of particles included in a biological liquid sample is measured by resistive pulse sensing in the step (a). The biological liquid sample may be a liquid sample, such as nasal mucus, saliva, urine, or blood of a test subject, or a liquid sample prepared by subjecting a solid sample, such as oral epithelium, skin, hair, or nail to, for example, crushing and dissolution in a liquid. Alternatively, the liquid sample may be obtained by subjecting any of the above liquid samples to a treatment such as dilution, concentration, or filtration (for example, filtration with a filter having an opening of 500 μm). The liquid sample to be measured is preferably in a state of a physiological buffer solution, and may be, for example, a PBS buffer solution such as ⅓×PBS, or a Tris buffer solution.

From the viewpoint of performing a rapid measurement in a medical institution, the treatment to be carried out, if any, is as simple as possible (for example, treatment including only filtration using a filter having an appropriate opening). In the present disclosure, even if there are many co-present substances, other than the detection target, in the liquid sample, the modified nanoparticle selectively attaches to the detection target owing to the oligosaccharide that selectively captures the detection target, and the dispersibility improving group further reduces non-specific attachment, as a result of which high sensitivity measurement is possible. The lower limit of the number of detection target particles necessary for imparting detectability is, for example, in a range of from 20 to 1000, or from 50 to 200. Such sensitivity is much higher than in conventionally performed detection using immunochromatography. Of course, there is no problem in measuring a greater number of particles than the lower limit, and about 500 to about 1000 particles may be measured. In the case of detection of an influenza virus, sufficient detection is enabled by measuring 1 hemagglutinin unit (HAU) or more of virus particles.

As described above, in the method of detecting a virus or bacterium according to the present disclosure, a pretreatment of a sample can be minimized, and a shift of an electrical resistance peak can automatically be detected by a computer. Therefore, it is possible to obtain a judgement result concerning the presence and/or amount of the detection target, for example, within 10 min. Also, a resistive pulse sensing device can be downsized, so that the resistive pulse sensing device can easily be installed in a medical institution.

In resistive pulse sensing, a peak at which the electric resistance value increases is observed when each particle passes through the pore of the membrane. Based on the height of this peak, the size of each particle can be determined, and a histogram of particle size distribution can be generated. It is preferable to perform measurements for calibration using standard samples for calibration that contain particles having known particle sizes. However, since the particle volume is proportional to the peak height in resistive pulse sensing, calibration may be carried out using only one standard sample for calibration rather than using plural standard samples. The particle size can be obtained as the diameter of a sphere corresponding to the obtained particle volume.

In the step (a), the biological liquid sample may be mixed with nanoparticles in which an oligosaccharide for selectively capturing the detection target is not present on their surfaces (nanoparticles that have the same configuration as that of the modified nanoparticle used in the step (b) except for lacking the oligosaccharide) before measurement by resistive pulse sensing is performed. In a case in which the detection target is mixed with nanoparticles in which an oligosaccharide that selectively captures the detection target is not present on their surfaces, free nanoparticles will also be included in the particle size distribution, and, therefore, comparison with a particle size distribution obtained in the step (c) becomes still easier.

In the step (b), the biological liquid sample is mixed with the reagent according to the present disclosure to obtain a mixed liquid. As a result of this mixing:

in a case in which the detection target is present in the biological liquid sample, the oligosaccharide in the reagent according to the present disclosure, which selectively captures the detection target, captures the detection target, and, as a result, the modified nanoparticle attaches to the detection target;

in a case in which the detection target is not present in the biological liquid sample, the modified nanoparticle remains free.

The measurement in the step (a) or the step (b) may be performed at ordinary temperature.

Mixing may be performed by manual stirring, or may be performed, for example, by applying vibrations using a vortex mixer or the like, stirring with a stirrer, or pipetting. There is no particular restriction on the mixing ratio between the biological liquid sample and the reagent according to the present disclosure. However, when the amount of the reagent according to the present disclosure is too small, detection of a change in size (size shift) would be difficult. Therefore, the mixing is preferably carried out under a condition that would provide a coverage by the modified nanoparticles of 50% or more (for example, from 50% to 99%) when the detection target is present in the biological liquid sample. Preferably, it is preferable to use at least 10 times as many modified nanoparticles as the estimated number of virus particles that would be present in a case in which the virus is present, and the number of the modified nanoparticles is more preferably at least 100 times the estimated number of virus particles, and further preferably at least 1000 times the estimated number of virus particles. By saturating the modified nanoparticles relative to the virus particles, the amount of change in the particle size is increased, so that clearer detection is enabled. For example, the estimated number of virus particles may be set in the order of 10⁹ particles/mL, and the number of modified nanoparticles may be set in the order of 10¹² particles/mL. Although there is no particular restriction on the upper limit of the ratio of the number of the modified nanoparticles to the estimated number of virus particles, the number of the modified nanoparticles may be, for example, 100,000 times the estimated number of virus particles or less.

In at least one of the step (a) or the step (b), the biological liquid sample may be mixed with an aqueous medium. When the viscosity of the biological liquid sample is high, or the particle concentration is high, it is advantageous to dilute the biological liquid sample with an aqueous medium from the viewpoint of improving the measurement accuracy. When the mixing of the biological liquid sample with an aqueous medium is performed, the mixing is preferably performed in both of the step (a) and the step (b).

In the step (c), the particle size distribution of particles contained in the mixed liquid is measured by resistive pulse sensing. The specifics of this step are the same as those of the step (a), except that the target to be measured is changed. As a result of this measurement, if the detection target is present in the biological liquid sample, comparison between the particle size distribution obtained in the step (a) and the particle size distribution obtained in the step (c) reveals that the peak of the virus or bacterium that is the detection target in the particle size distribution obtained in the step (a) has shifted toward the larger particle size side in the particle size distribution obtained in the step (c) due to attachment of the modified nanoparticle. When this shift is recognized, it can be judged that the virus or bacterium that is the detection target is present in the biological liquid sample. Meanwhile, the expression “particle size range corresponding to the virus or bacterium” as used herein refers to a range from the particle size of the virus or bacterium to the particle size of the virus or bacterium particle when the maximum possible amount of the modified nanoparticles have attached to the particle. The judgement as to the presence or absence of a peak shift is carried out in the “particle size range corresponding to the virus or bacterium”. Even if a peak shift is observed at a particle size outside the particle size range corresponding to the virus or bacterium, such a peak shift does not indicate the presence of the virus or particle. For example, observation as to whether a shift in the particle size distribution occurs may be performed in a range from [the particle size of the virus or bacterium itself] to [the particle size of the virus or bacterium+2×(average particle size of the modified nanoparticles)]. Further, with respect to the magnitude of the peak shift (increase in particle size), a cutoff value may be set based on the particle size of the reagent or the modified nanoparticle to be used. For example, the cutoff value may be set to a value that is from 10% to 70% of the average particle size of the modified nanoparticles. The magnitude of a shift of a peak may be determined based on the magnitude of the shift of the apex of the peak.

When an aggregate of plural bacterium or virus particles is included, the shape of a peak in the particle size distribution becomes complicated. In this case, the peak should be divided to plural peaks by waveform separation through fitting into plural normal distributions, and the shift of each peak should be examined.

In a case in which the detection target is not present in the biological liquid sample, comparison between the particle size distribution obtained in the step (a) and the particle size distribution obtained in the step (c) would reveal that the peak corresponding to the virus or bacterium that is the detection target in the particle size distribution obtained in the step (c) is found at the same particle size as that in the particle size distribution obtained in the step (a). Incidentally, the modified nanoparticles are also present in the particle size distribution obtained in the step (c). Therefore, in a case in which the modified nanoparticle is not added in the step (a), the particle size distribution obtained in the step (c) would also include a newly added peak corresponding to the modified nanoparticles in a free state.

Resistive pulse sensing enables measurement of the concentration of a specific virus or bacterium in the biological liquid sample by counting the number of particles included in the shifted peak, utilizing the ability of resistive pulse sensing to measure individual particles. In other words, resistive pulse sensing is not only capable of determination of the presence or absence of specific virus or bacterium, but also capable of quantitative measurement the number of the specific virus or bacterium. Conventional quantitative measurements of a specific virus or bacterium mainly involve measurement of the titer, and have no examples in which the number itself of a specific virus or bacterium was counted. The method according to the present disclosure is also novel in this context.

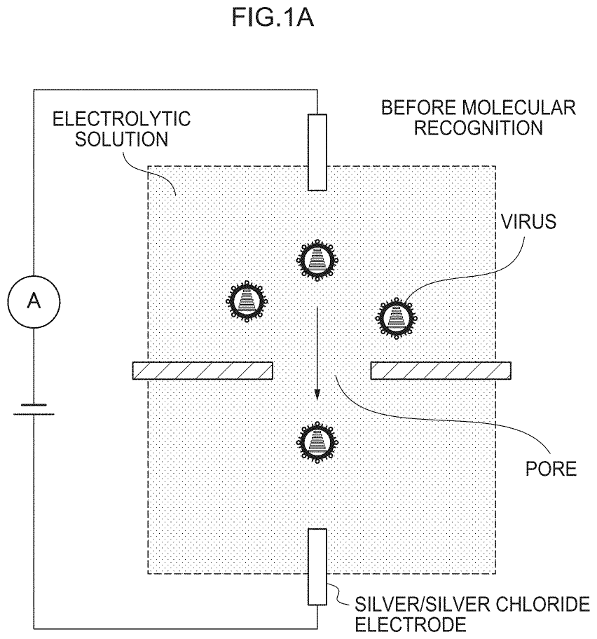

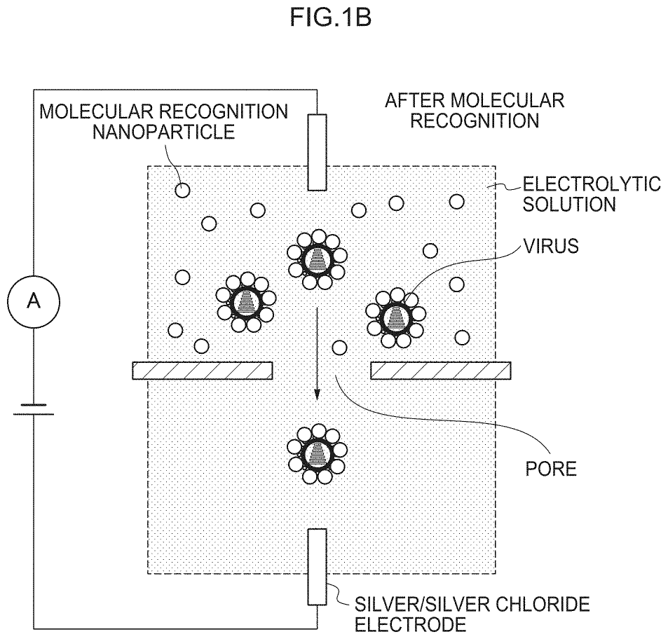

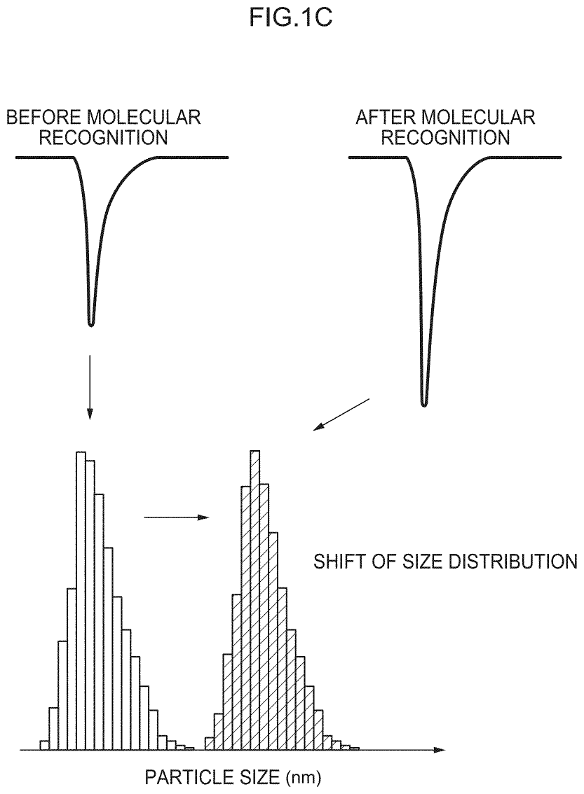

An example of resistive pulse sensing according to the present disclosure will be described with reference to the drawings. FIG. 1A is a schematic diagram illustrating a state in which a sample including virus particles is measured with a resistive pulse sensing device in the step (a). The resistive pulse sensing device shown in the figure includes two chambers filled with an electrolytic solution, a membrane serving as a partition between the chambers, a pore provided in the membrane, two electrodes configured to apply a voltage between both chambers, a power source configured to apply the voltage, and an ammeter configured to measure the electric current flowing therethrough. The voltage is applied between the first chamber and the second chamber provided on opposite sides of the membrane having the pore. A change in the electric current value occurring when the virus passes through the pore is measured by the ammeter, and the resistance value is monitored based on the measured value.

FIG. 1B is a schematic diagram illustrating a state in which virus particles having nanoparticles attached thereto are measured by resistive pulse sensing in the step (c), the nanoparticles (molecular recognition nanoparticles) having, on a surface thereof, an oligosaccharide that selectively captures a detection target. The particle sizes of the virus particle-nanoparticles complexes passing through the pore are increased as compared to the particle sizes of the virus particles alone.

FIG. 1C is a diagram illustrating that the peak corresponding to the virus particles has moved (shifted) toward the larger particle size side, from the peak corresponding to the virus particles in the particle size distribution obtained in the step (a) (before molecular recognition) to the peak corresponding to the virus particles in the particle size distribution obtained in the step (c) (after molecular recognition). In addition, the number of particles included in the peak can also be determined, and, based on the obtained number, the number of virus particles in the liquid sample can also be measured. When the detection target is not included in the liquid sample, such a shift does not occur.

As described above, according to the present disclosure, a virus or bacterium can selectively be detected at high sensitivity using modified nanoparticles having a surface to which an oligosaccharide that selectively captures a virus or bacterium is bound. This makes it possible to determine the presence of infection in a patient infected with a virus or bacterium at an earlier stage, and to start an appropriate treatment at an earlier stage.

### EXAMPLES

An embodiment of the present disclosure will be described hereinbelow more specifically by way of examples. However, the following examples do not limit embodiments according to the present disclosure.

### Reagents Used in Examples

11-Mercaptoundecanoic acid (hereinafter simply referred to as MUA) was purchased from Sigma-Aldrich Co.; Neu5Acα(2-6)Galβ(1-4)GlcNAc-β-ethylamine (6′-sialyl-N-acetyllactosamine-β-ethylamine: hereinafter simply referred to as 6′SLN) and Neu5Acα(2-3)Galβ(1-4)GlcNAc-β-ethylamine (3′-sialyl-N-acetyllactosamine-β-ethylamine: hereinafter simply referred to as 3′SLN) were purchased from Tokyo Chemical Industry Co., Ltd.; 4-(4,6-dimethoxy-1,3,5-triazin-2-yl)-4-methylmorpholinium chloride n-hydrate (hereinafter simply referred to as DMT-MM) was purchased from Wako Pure Chemical Industries, Ltd.; and N-(11-mercaptoundecyl)-N,N-dimethyl-3-ammonio-1-propanesulfonate (hereinafter simply referred to as SB-SH, where SB represents sulfobetaine, and SH represents thiol) was purchased from Dojindo Laboratories. These chemicals were used as they were purchased.

The structure of 6′SLN and the structure of 3′SLN are the structure of α2,6-sialyl-N-acetyllacsamine-β-ethylamine and the structure of α2,3-sialyl-N-acetyllacsamine-β-ethylamine, respectively, which are shown above.

A citric-acid-stabilized gold nanoparticle (diameter 20 nm) was purchased from Sigma-Aldrich Co. Dialysis was performed using a Spectra/Por® dialysis membrane (Biotech CE Dialysis Tubing, molecular weight cut-off: 3.5 kD, available from Spectrum Laboratories). The size distribution of an influenza A H1N1 solution was measured with a nanoparticle measuring device qNano (available from Izon Science Ltd.). A zeta potential was measured by ZETASIZER Nano ZS (available from Malvern Instruments). A human influenza virus type A subtype H1N1 (A/PR/8/34) was cultured in chick embryo, and inactivated by a 0.05% (weight/weight) formalin solution. The HA titer of the resulting inactivated influenza virus type A subtype H1N1 solution was 256 HAU.

### Preparation of MUA-GNP from Tetrachloroauric (III) Acid

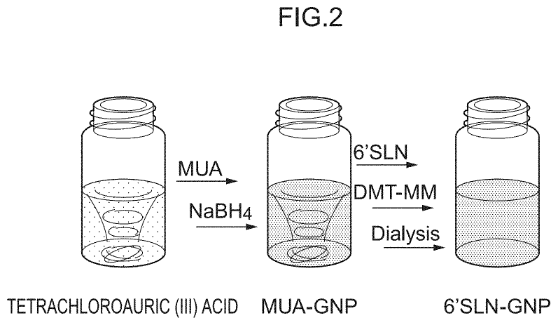

A 4.44 mM MUA solution was prepared using 0.1 M NaOH, and 20 mL of 1.42 mM tetrachloroauric (III) acid was mixed with 2 mL of the MUA solution. After 10 min, 350 μL of a 150 mM NaBH₄ solution was added very slowly. The solution was stirred overnight (reaction on the left side of FIG. 2).

In this operation for reducing a gold ion, small gold nanoclusters were formed and allowed to grow into nanoparticles. MUA molecules are firmly bonded to a gold surface by S—Au bonds to form a self-assembled monolayer (SAM). This layer maintains the suspension state of gold nanoparticles (hereinafter simply referred to as GNPs) and prevents further growth.

From the measurement results of the zeta potential and DLS, anionic GNPs on which MUA was immobilized and which had a diameter of 14.9 nm were obtained by this reaction (MUA-GNPs having a zeta potential of −38.0 mV and a polydispersity index of 0.267). From a transmission electron microscope (TEM) image, the Au core size was found to be about 10 nm.

### Preparation of 6′SLN-GNP and 3′SLN-GNP

First 3.04 μmol of 6′SLN or 3′SLN was dissolved in 4.8 mL of the MUA-GNP solution, and then 4.8 μL (3.38 μmol) of a 353 mM DMT-MM solution was added, and the solution was stirred at room temperature for 3 hours. The resultant was stored at 4° C. overnight. Excessive free SLN and by-products were removed by dialysis (reaction on the right side of FIG. 2). A schematic diagram of the surface modification of 6′SLN-GNP is shown below.

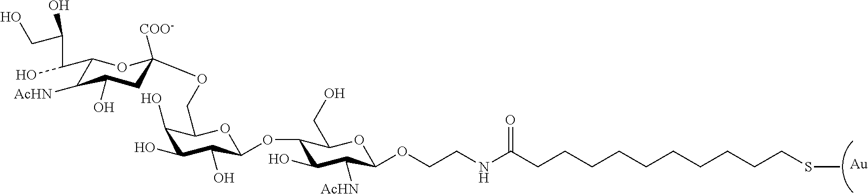

In this modification process with 6′SLN, 4-(4,6-dimethoxy-1,3,5-triazin-2-yl)-4-methylmorpholinium chloride n-hydrate (DMT-MM) was used as a condensing agent. After the reaction with 6′SLN, the color of the GNP solution had changed slightly (6′SLN-GNP, right side of FIG. 2). For explaining the details of this color change more clearly, the absorption spectra of MUA-GNPs and 6′SLN-GNPs were measured. The results thereof confirmed a shift of the surface plasmon peak toward red (the position of a maximum peak near 520 nm was 515 nm in the case of MUA-GNPs while the position was 521 nm in the case of 6′SLN-GNPs), which derived from a refractive index change caused by formation of a 6′SLN layer on the GNPs. The zeta potential had changed from −38.0 mV to −24.6 mV after the modification with 6′SLN. This result also verifies fixation of 6′SLN. This is because MUA-GNP has a densely packed COO⁻ surface, while 6′SLN-GNP has bulky sialic acid on the surface. Therefore, the charge density on the GNP surface had decreased. From the above results, it was confirmed that 6′SLN was certainly fixed to the GNPs by a condensation reaction. A similar result was also obtained in the case of modification with 3′SLN.

### Preparation of SB-GNP, 6′SLN/SB-GNP, and 3′SLN/SB-GNP

10 μL of the citric-acid-stabilized GNPs (1OD, 6.54×10¹¹ particles/mL) was mixed with 2 mL of a 1 mM SB-SH solution containing 0.1 M NaOH, and the solution was stirred overnight. The solution was concentrated to 5 mL by evaporation under reduced pressure, and dialyzed three times with 500 mL of pure water (for 2 hours, 4 hours, and 12 hours, respectively). The SB-GNP solution obtained was stored at 4° C.

In the uniform GNPs suspended in citric acid used in this experiment, citric acid interacts with the gold surface by physical adsorption, and the citric acid layer maintains the dispersion stability of the GNPs. Since the physical adsorption of citric acid is easily exchanged by a strong S—Au bond, this layer can easily be exchanged by a molecule having a terminal SH.

10 mL of the citric-acid-stabilized GNPs (1OD, 6.54×10¹¹ particles/mL) was mixed with 1 mL of a 1 mM SB-SH solution containing 0.1 M NaOH and 1 mL of a 1 mM MUA solution containing 0.1 M NaOH, and the solution was stirred overnight. The solution was concentrated to 5 mL by evaporation under reduced pressure, and dialyzed three times with 500 mL of pure water (for 2 hours, 4 hours, and 12 hours, respectively). The SB/MUA-GNP solution obtained was stored at 4° C. A schematic diagram of the surface modification of SB/MUA-GNP is shown below.

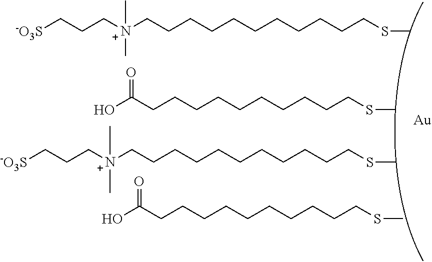

A half of the SB/MUA-GNP solution was mixed with 0.25 mL of a 4 mM 6′SLN solution, and the other half of the SB-MUA-GNP solution was mixed with 0.25 mL of a 4 mM 3′SLN solution. To each of the resultant solutions, 0.25 mL of a 4 mM DMT-MM solution was added, and the solutions were stirred at room temperature for 3 hours. After the condensation reaction, the solutions were stored at 4° C. overnight. These solutions were dialyzed with 500 mL of pure water to remove excessive 6′SLN or 3′SLN and by-products (for 2 hours, 4 hours and 12 hours, respectively). The 6′SLN/SB-GNPs and 3′SLN/SB-GNPs obtained were stored at 4° C. As a control experiment, SB-GNPs were also exposed to 6′SLN and DMT-MM, following the same procedure.

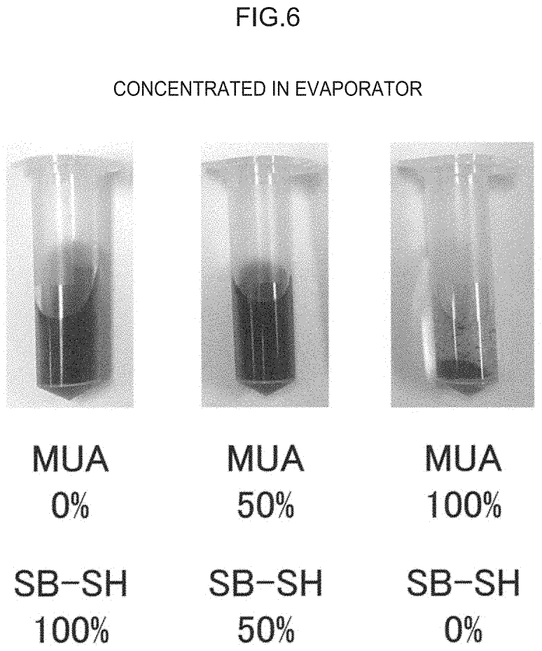

In order to realize the improvement of dispersion stability, the obtained nanoparticle solutions were concentrated using a rotary evaporator. MUA-GNPs irreversibly aggregated after the concentration, but MUA/SB-GNPs maintained dispersion stability (FIG. 6). SB-GNPs also maintained dispersion stability. In this regard, although MUA-GNPs electrostatically disperse in water due to negative charges and maintain dispersion stability, an increase in salt concentration during concentration in an evaporator shields the ion electric field, and weakens electrostatic repulsion. As a result of this mechanism and an increase in the frequency of mutual collisions of MUA-GNPs due to the concentration, aggregation due to hydrophobic interaction was observed in MUA-GNPs. Meanwhile, GNPs to which SB-SH was fixed maintained dispersion stability even though negative charges were shielded by a citrate buffer. This is conceivably because aggregation by hydrophobic interactions was reduced due to formation of a quite firm hydrated surface when the surface has a betaine structure.

### Resistive Pulse Sensing of Virus with 6′SLN-GNP

A solution of influenza virus type A subtype H1N1 was diluted to 1 HAU in a ⅓ PBS buffer. A solution of 6′SLN-GNPs in a ⅓ PBS buffer (1.52 OD, 2.49×10¹² particles/mL) was also prepared. The solutions, each in an amount of 45 μL, were mixed, allowed to stand for 10 min, and measured by resistive pulse sensing using qNANO (trade name) manufactured by Izon Science Ltd. Measurement of the virus alone and measurement of the virus using 3′SLN-GNPs were also performed under the same conditions as control experiments.

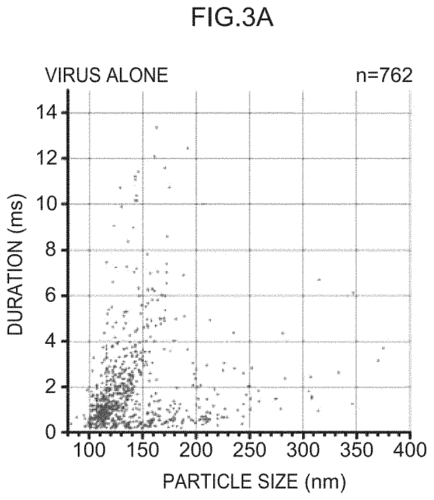

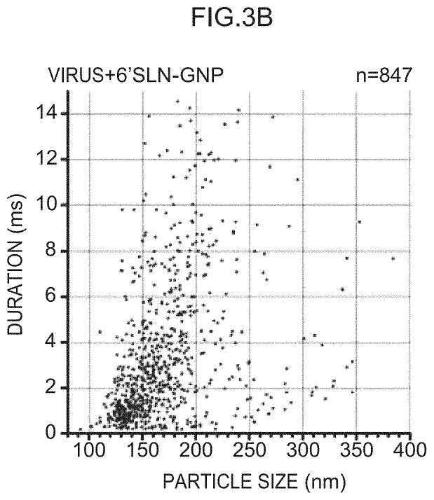

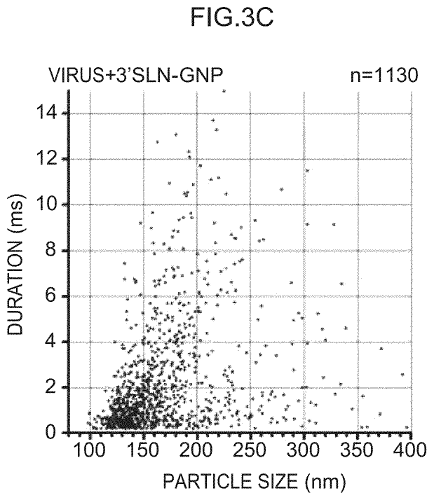

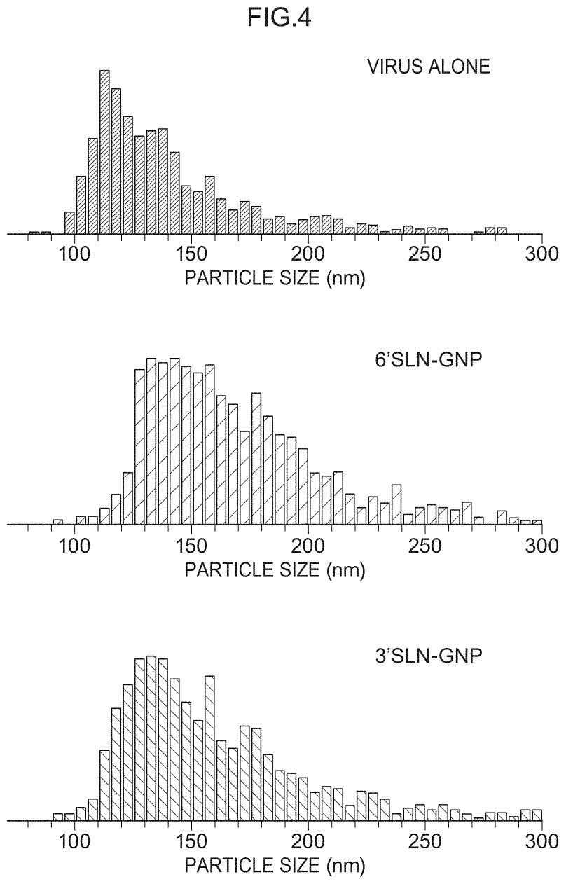

Specifically, after the confirmation of the identity of 6′SLN-GNPs, resistive pulse sensing measurement was conducted in order to confirm the interaction with influenza virus type A subtype H1N1. FIG. 3A to FIG. 3C are scatter plots of duration vs. particle size obtained from the resistive pulse sensing measurement results. FIG. 3A is a scatter plot of duration vs. particle size obtained from the results of resistive pulse sensing measurement of the virus solution. FIG. 3B is a scatter plot of duration vs. particle size obtained from the results of resistive pulse sensing measurement in a case in which 6′SLN-GNPs were mixed with the virus solution. FIG. 3C is a scatter plot of duration vs. particle size obtained from the results of resistive pulse sensing measurement in a case in which 3′SLN-GNPs were mixed with the virus solution. These results indicate that the size distribution of the virus shifted toward the larger particle size side after mixing with 6′SLN-GNPs. This indicates an interaction between the virus and 6′SLN-GNPs. In the size histograms (FIG. 4) obtained by converting the scatter plots, the shift in size distribution due to attachment of GNPs to the virus can be understood more clearly. However, a size shift was also observed when the virus was mixed with 3′SLN-GNP, although the size shift was not as large as that in the case of 6′SLN-GNP. This indicates that a non-specific interaction occurred between the virus and 3′SLN-GNPs. It cannot be confirmed from the comparison of these data whether or not the size shift was due to a specific interaction.

There is possibility that undesired attachment of 3′SLN-GNPs to the virus occurred due to low dispersion stability of the obtained 6′SLN-GNPs and 3′SLN-GNPs. In the case of 3′SLN-GNPs or 6′SLN-GNPs, the particles are in the state of suspension due to electrostatic interaction. It is conceivable that there is a possibility that charges are shielded in the ⅓×PBS condition, and that the shielding of charges caused the undesired attachment due to an intermolecular force such as a hydrophobic interaction, a hydrogen bond or a van der Waals force. Furthermore, the solution of influenza virus type A subtype H1N1 was collected from the chorioallantoic fluid of an infected chick embryo. The fluid contains many impurities, such as proteins, and there is a possibility that these impurities promote undesired aggregation of GNPs. However, the magnitude of the distribution shift when the virus was mixed with 3′SLN-GNPs was smaller than that in the case with 6′SLN-GNPs. If the non-specific interaction between the virus and GNPs is reduced, the specific recognition by 6′SLN-GNPs will be observed more clearly.

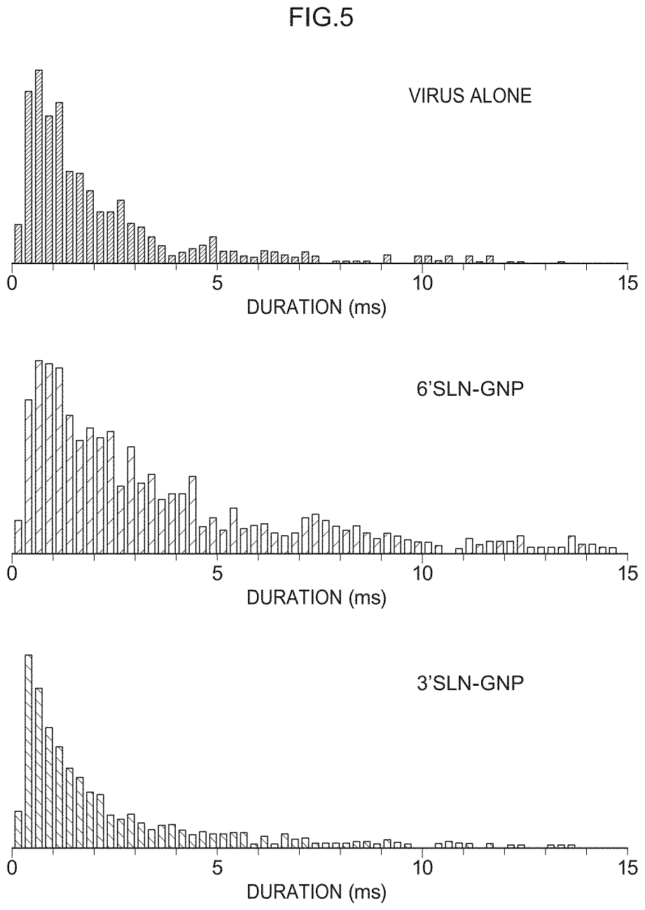

Histograms of duration (duration of an electrical resistance increase peak) were compared for reference (FIG. 5), but there was no correlation between the distribution shift and the duration.

### Resistive Pulse Sensing of Virus with 6′SLN/SB-GNP

A solution of influenza virus type A subtype H1N1 was diluted with ⅓ PBS buffer to 2 HAU. A solution of 6′SLN-GNPs in ⅓ PBS buffer (1.29 OD, 8.41×10¹¹ particles/mL) was also prepared. The solutions, each in an amount of 45 μL, were mixed, allowed to stand for 10 min, and measured by resistive pulse sensing (the final concentration of the virus was 1 HAU (8.61×10⁸ particles/mL)). Measurement of the virus alone, measurement of the virus with SB-GNPs, and measurement of the virus with 6′SLN-GNPs were performed under the same conditions as control experiments.

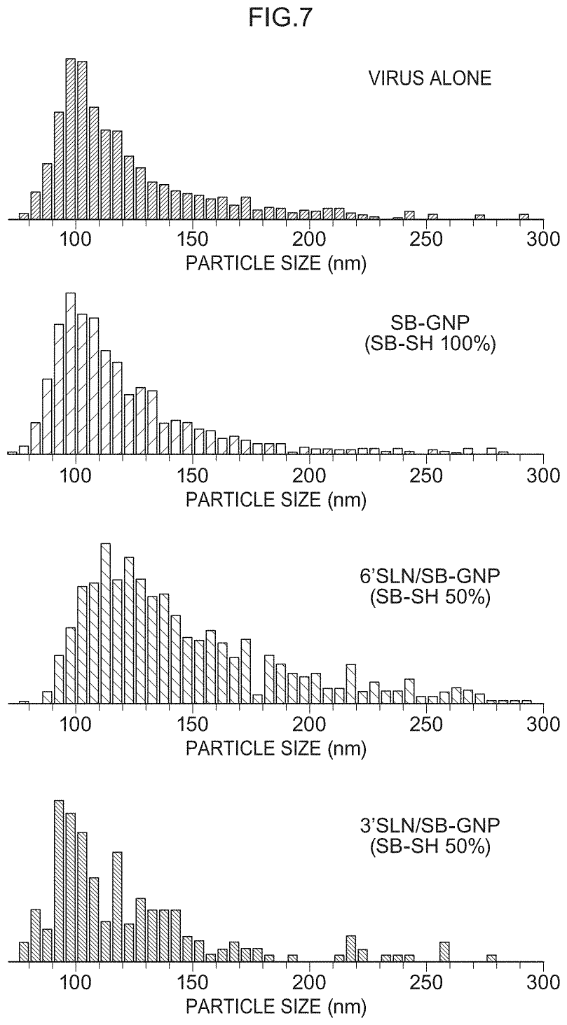

The results of molecular recognition experiments for evaluating measurement performance were as follows. There was no recognizable change in the virus size distribution in the case of the virus with SB-GNPs (without SA receptor) compared with the virus solution (the top graph and the second graph from the top in FIG. 7). These results indicate that nonspecific adsorption of GNPs to the virus was completely suppressed due to improvement of the dispersion stability. Meanwhile, the size distribution in the case of the virus solution containing 6′SLN/SB-GNPs showed a clear shift toward the larger particle size side (the third graph from the top in FIG. 7). This result indicates that the interaction between the virus and GNPs is promoted by the 6′SLN moiety. In order to confirm the specificity of this interaction, the size distribution in the case of 3′SLN/SB-GNPs was also evaluated as a control experiment, as a result of which no shift of the distribution was observed (the bottom graph in FIG. 7). From these results, we concluded that non-specific interaction was suppressed by using a SB-SH co-immobilization technique.

This detection result is more sensitive than ICT. According to the aforementioned report on the immunochromatography (ICT) technique for discriminating avian influenza or human influenza, positive results were observed from 32 HAU. HAU stands for “hemagglutination unit”, and this value is defined as infectivity of virus when chick erythroid cells are used. In the present experimental results, a size shift was already observed using a 1 HAU virus solution, and this concentration was lower than that required in the ICT technique. According to our experiments, 567 virus particles passed through the pore within 10 min. Since a size distribution histogram sufficient for detecting a size shift is created by this number of virus particles, it can be presumed that detection of virus particles will certainly be possible, even if the number of measured particles is further reduced. If statistical information can be obtained with 100 virus particles, the sensitivity will be 0.2 HAU or more. Further, it can be expected that higher sensitivity can be achieved by further optimizing the experimental conditions based on the results of these experiments. Thus, the resistive pulse sensing measurement using a molecular recognition technique is a promising technique for accurately detecting target particles.

### Quantitative Calculation of Viruses

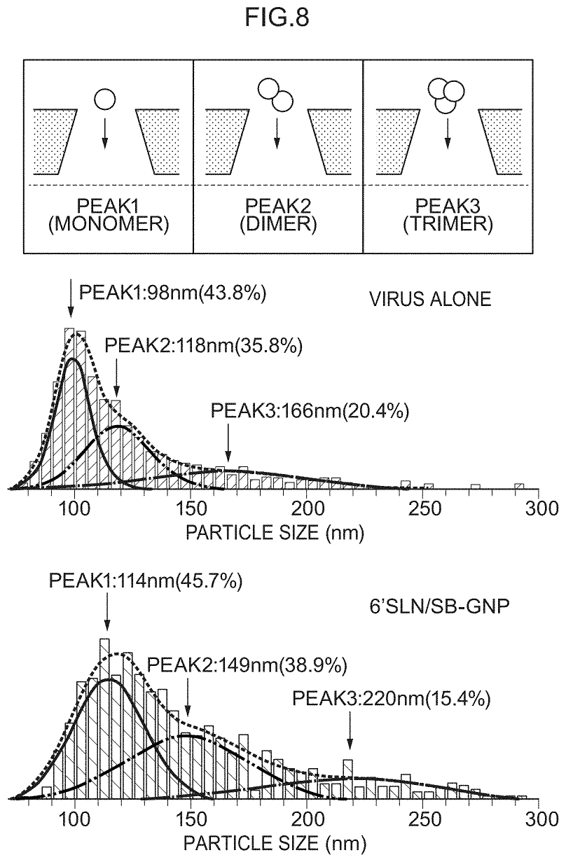

In our experiments, the obtained virus size distributions were asymmetric in all cases, due to the presence of aggregated virus. Since the virus size is from 80 to 120 nm in diameter, there is no doubt that the peak in the distribution is from monodisperse influenza virus. However, the virus is not always suspended in a solution in an independently dispersed state, and may be dispersed in an aggregated state. For this reason, we calculated the amount of the virus quantitatively, assuming that the size distribution was the sum of monomers, dimers, and trimers. FIG. 8 shows three Gaussian distribution curves derived from monomers, dimers, and trimers, which were obtained by waveform separation of the size distribution. According to this calculation, peaks at 98 nm, 118 nm, and 166 nm, respectively, were obtained in the size distribution. The dimer and trimer peaks in the size distribution are obtained each as an average hydrodynamic radius. For example, when a dimer aligned perpendicularly to the pore plane passes, the electric current blockade is almost the same as that in the case of a virus monomer particle, and, when a dimer aligned parallel to the pore plane passes, the electric current blockade becomes maximum. In other words, the magnitude of the electric current blockade is influenced by the area of the cross-section that blocks the ionic current. This dependence on the particle direction is caused by the fact that the cross-sectional area of the pore is not constant in the depth direction (in this example, a conical shape). The large widths observed in the obtained size distribution were caused by the directions of the virus particles as they pass through the pore. The result of the waveform separation revealed that 43.8% by number of the particles were virus monomer particles, 35.8% were dimer particles, and 20.4% were trimer particles.

After the treatment with 6′SLN/SB-GNPs, all the peaks in the distribution shifted, and the ratio between the particle contents was in good agreement with the ratio in the case with virus alone. Therefore, the distribution change using molecular recognition is reliable information for detecting the target virus. The advantage of the resistive pulse sensing measurement is that quantitative calculation can be made easily from direct counting of virus particles. In most cases, the quantitative calculation of a virus in the medical field is not made in terms of a physical quantity of virus, but in terms of infectious titers such as hemagglutination unit (HAU), plaque-forming units (pfu/mL), and 50% tissue culture infectious dose (TCID 50/mL). These values do not represent the number of virus particles, but are well suited for measuring an infection risk. The infectivity of a virus depends on the type of virus, that is, the resistive pulse sensing measurement technique is useful for understanding infectivity by comparing a physical quantity and a virus titer. A numerical measurement may be able to create a novel technique for examining an immunological system from a different viewpoint from the conventional knowledge.

As shown from the above experimental results, when detection of a detection target was performed using a modified nanoparticle according to the present disclosure, selective detection of the detection target could be performed, distinguishing highly similar detection targets such as human influenza and an avian influenza. In doing so, the detection sensitivity was also high. In this regard, the human influenza virus type A subtype H1N1 (A/PR/8/34) used in the experiment was obtained by culturing in a chick embryo, and therefore contained a large amount of co-existing substances. However, even with respect to a biological sample with such a large amount of co-existing substances, selective detection with high sensitivity was possible. Further, although the sample was used after dilution in this experiment, use of the sample without dilution would enable detection with an even smaller amount.

Although 6′SLN was used for detection of human influenza in the above examples, a reagent or modified nanoparticle for detecting another virus or bacterium can easily be obtained by replacing 6′SLN with another oligosaccharide.

The disclosure of Japanese Patent Application No. 2017-090567, filed Apr. 28, 2017, is incorporated herein by reference in its entirety

All publications, patent applications, and technical standards mentioned in this specification are incorporated herein by reference to the same extent as if each individual publication, patent application, or technical standard was specifically and individually indicated to be incorporated by reference.

1. A modified nanoparticle, comprising:
nanoparticle;
a dispersibility improving group bound to a surface of the nanoparticle; and
an oligosaccharide that is bound to the surface of the nanoparticle, and that selectively captures a specific virus or bacterium.

2. The modified nanoparticle according to claim 1, wherein the nanoparticle is a metal nanoparticle or a polymer nanoparticle.

3. The modified nanoparticle according to claim 1, wherein a number average particle size of the nanoparticle is from 5 nm to 100 nm.

4. The modified nanoparticle according to claim 1, wherein the oligosaccharide selectively captures an influenza virus.

5. The modified nanoparticle according to claim 4, wherein the oligosaccharide selectively captures a specific type of influenza virus.

6. The modified nanoparticle according to claim 1, wherein the dispersibility improving group has, at a terminal thereof, a sulfobetaine group, a carboxybetaine group, or a phosphobetaine group.

7. A dispersion liquid, comprising the modified nanoparticle according to claim 1, and an aqueous medium.

8. A set for resistive pulse sensing, the set comprising the modified nanoparticle according to claim 1, or the dispersion liquid according to claim 7, and a pore-containing membrane for resistive pulse sensing.

9. A set for detection of a specific virus or bacterium, the set comprising the modified nanoparticle according to claim 1 or the dispersion liquid according to claim 7, and a resistive pulse sensing device.

10. A reagent for detection of a specific virus or bacterium by resistive pulse sensing, the reagent comprising the modified nanoparticle according to claim 1.

11. The reagent according to claim 10, for detecting the specific virus or bacterium based on presence or absence of a shift of a particle size peak in a particle size distribution.

12. A method of detecting a virus or bacterium, the method comprising:
(a) a step of measuring a particle size distribution of particles included in a biological liquid sample by resistive pulse sensing;
(b) a step of preparing a mixed liquid by mixing the biological liquid sample with the reagent according to claim 10; and
(c) a step of measuring a particle size distribution of particles included in the mixed liquid by resistive pulse sensing,
wherein the biological liquid sample is judged to include the virus or bacterium when there is a peak in a particle size range corresponding to the virus or bacterium, of which a peak position in the particle size distribution obtained in the step (c) exhibits a shift toward a larger particle size side, compared to a peak position thereof in the particle size distribution measured in the step (a).

13. The detection method according to claim 12, wherein the biological liquid sample is mixed with a nanoparticle not having the oligosaccharide on a surface thereof, before the measurement by resistive pulse sensing in the step (a).

14. The detection method according to claim 12, wherein the biological liquid sample is mixed with an aqueous medium in at least one of the step (a) or the step (b).
